(19) United States
(12) Patent Application Publication
(10) Pub. No.: US 2005/0153911 A1
(43) Pub. Date: Jul. 14, 2005

(54) Antisense modulation of extracellular-signal-regulated kinase-6 expression

(75) Inventors: Frank Bennett, Carlsbad, CA (US); Kenneth W. Dobie, Del Mar, CA (US); Eric G. Marcusson, San Diego, CA (US)

Correspondence Address: MARY E. BAK, HOWSON AND HOWSON, SPRING HOUSE CORPORATE CENTER, BOX 457, SPRING HOUSE, PA, 19477, US

(73) Assignee: ISIS Pharmaceuticals, Inc, Carlsbad, CA (US)

(21) Appl. No.: 10/516,505

(22) PCT Filed: Jun. 16, 2003
(86) PCT No.: PCT/US03/16214

(51) Int. Cl. A61K 48/00; C07H 21/02

(52) U.S. Cl. 514/44; 536/23.1

(57) ABSTRACT

Antisense compounds, compositions and methods are provided for modulating the expression of extracellular-signal-regulated kinase-6. The compositions comprise antisense compounds, particularly antisense oligonucleotides, targeted to nucleic acids encoding extracellular-signal-regulated kinase-6. Methods of using these compounds for modulation of extracellular-signal-regulated kinase-6 expression and for treatment of diseases associated with expression of extracellular-signal-regulated kinase-6 are provided.

### FIELD OF THE INVENTION

The present invention provides compositions and methods for modulating the expression of extracellular-signal-regulated kinase-6. In particular, this invention relates to compounds, particularly oligonucleotides, specifically hybridizable with nucleic acids encoding extracellular-signal-regulated kinase-6. Such compounds have been shown to modulate the expression of extracellular-signal-regulated kinase-6.

### BACKGROUND OF THE INVENTION

Mitogen-activated protein kinases (MAPKs) are a family of serine/threonine protein kinases that play a crucial role in relaying extracellular signals transduced through the plasma membrane to the nucleus. MAPKs respond to cues, such as cellular stresses and pro-inflammatory cytokines, mitogens and tumor-promoting phorbol esters, and hormones or |polypeptide growth factors. MAPKs respond by promoting changes in gene expression, cell proliferation, and cell survival during embryogenesis, growth and differentiation. The MAPK pathway includes three major families: the extracellular-signal-regulated kinases (ERKs), the c-Jun NH₂-terminal kinases (JNKs)/stress-activated protein kinases (SAPKs), and the p38 kinases (English and Cobb, Trends Pharm. Sci., 2002, 23, 40-45).

Interest in MAPKs as therapeutic targets has soared since the identification of potential pharmacological inhibitors of the MAPKs ERK1, ERK2, two p38 isoforms, three JNK/SAPKs, and ERK5. In particular, pyridinyl imidazole anti-inflammatory drugs have been shown to target p38 MAPK and demonstrate efficacy in treatment of arthritis and psoriasis, reaffirming the idea that intracellular enzymes with multiple functions are valuable therapeutic targets for specific applications. Thus, most extensive activity in MAPK inhibitor development has revolved around p38 MAPKs (English and Cobb, Trends Pharm. Sci., 2002, 23, 40-45).

In mammals, four major p38 MAPK isoforms have bee identified: p38α (SAPK2a), p38β2 (SAPK2b), p38γ (SAPK4), and extracellular-signal-regulated kinase-6 (also known as p38γ, ERK-6, ERK6, ERK5, ERK3, p38gamma, stress-activated protein kinase 3, SAPK3, mitogen-activated protein kinase 3, mitogen-activated protein kinase 12, MAPK12 and PRKM12). These isoforms appear to mediate distinct functions in vivo due, in part, to differences in their activation by the selective formation of functional complexes with a particular MAPK kinase, as well as to phosphorylation of p38-specific substrates (Enslen et al., EMBO J., 2000, 19, 1301-1311)

As the third member of the p38 group of MAP kinases, the extracellular-signal-regulated kinase-6 gene was identified from two overlapping expressed sequence tag (EST) clones in the GenBank database. While other p38 family members are expressed in multiple tissues, extracellular-signal-regulated kinase-6 mRNA was expressed primarily in skeletal muscle. Thus, extracellular-signal-regulated kinase-6 is predicted to have unique functions and substrate preferences (Li et al., Biochem. Biophys. Res. Commun., 1996, 228, 334-340). By fluorescence in situ hybridization, the extracellular-signal-regulated kinase-6 gene was mapped to human chromosomal locus 22q13.3. Two cDNAs representing human extracellular-signal-regulated kinase-6 were identified, suggesting the existence of alternative splice forms that produce at least two protein isoforms, 357 and 367 amino acids in length. Although mRNA levels were found to be highest in skeletal muscle, extracellular-signal-regulated kinase-6 mRNA was also detected in other tissues (Goedert et al., Genomics, 1997, 41, 501-502).

Independently, the human extracellular-signal-regulated kinase-6 gene was isolated and the encoded protein reported to be a tissue-specific, signal-transducing factor connected to phosphotyrosine-mediated signaling pathways active during myogenic differentiation. Overexpression of extracellular-signal-regulated kinase-6 was found to enhance differentiation of myoblasts to myotubes. Inactivation inhibited this differentiation, maintaining cells in a proliferative state (Lechner et al., Proc. Natl. Acad. Sci. U. S. A., 1996, 93, 4355-4359).

Prolonged strenuous exercise, for example marathon running, results in a robust but transient increase in phosphorylation and activation of JNK and extracellular-signal-regulated kinase-6. This suggests that stress-activated protein kinases may be responsible for skeletal muscle adaptations to exercise (Boppart et al., J. Physiol., 2000, 526 Pt 3, 663-669). Hypoxia is a common physiological stress involved in a variety of pathological processes including angiogenesis, tumor progression and apoptosis. Extracellular-signal-regulated kinase-6 has is activated by hypoxia. Two D2-dopamine receptor antagonists have been shown to enhance hypoxia-induced phosphorylation of extracellular-signal-regulated kinase-6 in neural-like pheochromocytoma (PC12) cells derived from rat adrenal medullary tumors. Removal of extracellular calcium blocked the hypoxia-induced increase in extracellular-signal-regulated kinase-6 activity. Thus, the D2 receptor and calcium regulate extracellular-signal-regulated kinase-6 following hypoxic exposure (Conrad et al., Cell Signal., 2000, 12, 463-467).

A large family of G protein-coupled receptors related to the ml class of human muscarinic acetylcholine receptors can activate the c-Jun promoter via elevation of the activity of extracellular-signal-regulated kinase-6. Moreover, activation of extracellular-signal-regulated kinase-6 is necessary for full activation of the c-Jun promoter, apparently stimulating distinct regulatory elements within the promoter (Marinissen et al., Mol. Cell. Biol., 1999, 19, 4289-4301).

Small GTP-binding proteins of the Rho family can also initiate a cascade of signaling events that lead to the activation of extracellular-signal-regulated kinase-6. An activated form of the RhoA GTPase was found to potently stimulate activating phosphorylation of extracellular-signal-regulated kinase-6 by the lipid-activated protein serine/threonine kinase PRK1/PKN, leading to increased gene expression in NIH 3T3 murine fibroblasts (Marinissen et al., Genes Dev., 2001, 15, 535-553). Recently, the GTP-binding protein Rit was identified as a novel branch of the Ras family of G proteins. When overexpressed, an activated Rit mutant can efficiently transform NIH 3T3 cells and stimulate extracellular-signal-regulated kinase-6. Furthermore, extracellular-signal-regulated kinase-6 activation is required for the ability of Rit to stimulate gene expression and cellular transformation (Sakabe et al., FEBS Lett., 2002, 511, 15-20). Thus, the MKK3/MKK6-extracellular-signal-regulated kinase-6 pathway appears to regulate the gene expression and transforming abilities of the Rho and Rit Ras-like GTPases.

Extracellular-signal-regulated kinase-6 has been implicated in the cellular response to genotoxic agents widely used in the treatment of cancer, such as cisplatin or etoposide. In human melanoma cells, extracellular-signal-regulated kinase-6 is activated in response to cisplatin exposure or UV treatment. Etoposide causes a much more discrete phosphorylation than either of the other DNA-damaging agents. Genotoxic stresses appear to differentially activate several p38 isoforms, and the cellular response is believed to depend on cell type as well as the type of DNA damage (Pillaire et al., Biochem. Biophys. Res. Commun., 2000, 278, 724-728).

The MAPK pathway and extracellular-signal-regulated kinase-6 participate in the DNA-damage checkpoint. Activation of extracellular-signal-regulated kinase-6 is required for y-irradiation-induced G2 arrest of the cell cycle. Human fibroblasts expressing a dominant negative allele of extracellular-signal-regulated kinase-6 display an attenuated arrest which allows cells to escape the DNA-damage-induced checkpoint-mediated G2 delay. This interferes with the extracellular-signal-regulated kinase-6 pathway and dramatically enhances γ-irradiation-mediated cell killing. Extracellular-signal-regulated kinase-6 is, therefore, a potential target for improving cancer treatments by inactivating the G2/M arrest correlated with radioresistance |often acquired by tumor cells during cancer treatment (Wang et al., Mol. Cell. Biol., 2000, 20, 4543-4552).

In Alzheimer's disease, paired helical filaments (PHFs), consisting of the hyperphosphorylated form of the microtubule-associated protein tau, are the major fibrous element of neurofibrillary lesions. Extracellular-signal-regulated kinase-6 phosphorylates the tau protein at many sites known to be hyperphosphorylated in PHF-tau. Phosphorylation of tau resulted in a marked reduction in its ability to promote microtubule assembly, suggesting a possible role for extracellular-signal-regulated kinase-6 in Alzheimer's disease and other neurodegenerative disorders (Goedert et al., FEBS Lett., 1997, 409, 57-62).

Interestingly, extracellular-signal-regulated kinase-6 was found to be widely expressed at high levels in adult mouse brain. In subcellular localization studies, extracellular-signal-regulated kinase-6 was detected mainly in the cytoplasm and dendrites of neurons. Extraordinarily high basal activity in brain (as compared to peripheral organs), of all SAPK proteins, including extracellular-signal-regulated kinase-6, indicated that the SAPK pathway may be involved in normal brain physiology, as well as synaptic plasticity or synaptic remodeling (Lee et al., Brain Res. Mol. Brain Res., 1999, 70, 116-124).

U.S. Pat. No. 5,459,036 refers to an isolated nucleic acid molecule encoding the extracellular-signal-regulated kinase-6 polypeptide, and a nucleic acid probe able to detect the presence of extracellular-signal-regulated kinase-6 nucleic acid in a sample comprising an isolated nucleic acid molecule having at least 27 contiguous nucleotides of said nucleic acid. The patent also refers to a method of and a kit for detecting the presence of extracellular-signal-regulated kinase-6 RNA, an isolated nucleic acid molecule comprising a promoter effective to initiate transcription in a host cell and a nucleic acid encoding at least 9 contiguous amino acids of said polypeptide, an isolated nucleic acid molecule consisting of a promoter region and a sequence complimentary to said RNA and a transcription termination region functional in said cell. A transformed cell that contains the isolated nucleic acid molecule is also disclosed. Antisense extracellular-signal-regulated kinase-6 nucleic acid constructs are generally disclosed (Lechner et al., 1995).

European Patent EP 1174129 refers to the use of an active agent for influencing, particularly inhibiting, the expression of matrix-metalloproteases in eukaryotic cells, for the preparation of a medicament or a pharmaceutical composition for the treatment of cancer. A method for the treatment of cancer is described in which eukaryotic cells are treated by an active agent which influences, particularly inhibits, the expression of matrix-metalloproteases. The use or method is further characterized by targeting the active agent against at least one member of the matrix-metalloprotease signal transduction pathway. That member is a member of the p38 protein family, and that member may be extracellular-signal-regulated kinase-6. A pharmaceutical composition, wherein said active agent is influencing, preferably inhibiting, the expression of matrix-metalloproteases in eukaryotic cells is also disclosed, and optionally further comprises a pharmaceutically acceptable carrier (Zenner and Simon, 2000).

Currently, there are no known therapeutic agents that effectively inhibit the synthesis of extracellular-signal-regulated kinase-6. To date, investigative strategies aimed at modulating extracellular-signal-regulated kinase-6 function have involved the use of a small molecule inhibitor.

A potential anti-inflammatory small-molecule inhibitor of MAPK, VX-745, is under development for the treatment of rheumatoid arthritis (RA). VX-745 was reported to be active against several isotypes of p38 MAPK, including p38alpha, p38beta and extracellular-signal-regulated kinase-6. VX-745 was associated with the suppression of the release of inflammatory mediators, including interleukin (IL)-1beta and tumor necrosis factor (TNF)alpha, known to be implicated in exacerbating the pathophysiology of RA (Haddad, Curr. Opin. Investig. Drugs, 2001, 2, 1070-1076). However, high concentrations of VX-745 must be used to inhibit extracellular-signal-regulated kinase-6, and this inhibition is not specific for extracellular-signal-regulated kinase-6. It has effects on other kinases not in the MAPK family (English and Cobb, Trends Pharm. Sci., 2002, 23, 40-45).

Consequently, there remains a long felt need for additional agents capable of effectively inhibiting extracellular-signal-regulated kinase-6 function.

### SUMMARY OF THE INVENTION

The present invention provides compositions and methods for modulating extracellular-signal-regulated kinase-6 expression.

The present invention is directed to compounds, particularly antisense oligonucleotides, which are targeted to a nucleic acid encoding extracellular-signal-regulated kinase-6, and which modulate the expression of extracellular-signal-regulated kinase-6. Pharmaceutical and other compositions comprising the compounds of the invention are also provided. Further provided are methods of modulating the expression of extracellular-signal-regulated kinase-6 in cells or tissues comprising contacting said cells or tissues with one or more of the antisense compounds or compositions of the invention. Further provided are methods of treating an animal, particularly a mammal, and more particularly a human, suspected of having or being prone to a disease or condition associated with expression of extracellular-signal-regulated kinase-6 by administering a therapeutically or prophylactically effective amount of one or more of the antisense compounds or compositions of the invention.

### DETAILED DESCRIPTION OF THE INVENTION

The present invention employs oligomeric compounds, particularly antisense oligonucleotides, for use in modulating the function of nucleic acid molecules encoding extracellular-signal-regulated kinase-6, ultimately modulating the amount of extracellular-signal-regulated kinase-6 produced. Antisense technology is emerging as an effective means for reducing the expression of specific gene products and is uniquely useful in a number of therapeutic, diagnostic, and research applications for the modulation of extracellular-signal-regulated kinase-6 expression.

Antisense compounds are provided herein that specifically hybridize with one or more nucleic acids encoding extracellular-signal-regulated kinase-6. As used herein, the terms “target nucleic acid” and “nucleic acid encoding extracellular-signal-regulated kinase-6” encompass DNA encoding extracellular-signal-regulated kinase-6, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds that specifically hybridize to it is generally referred to as “antisense”. The functions of DNA to be interfered with include replication and transcription. The functions of RNA to be interfered with include all vital functions such as, for example, translocation of the RNA to the site of protein translation, translocation of the RNA to sites within the cell which are distant from the site of RNA synthesis, translation of protein from the RNA, splicing of the RNA to yield one or more mRNA species, and catalytic activity which may be engaged in or facilitated by the RNA.

The overall effect of such interference with target nucleic acid function is modulation of the expression of extracellular-signal-regulated kinase-6. In the context of the present invention, “modulation” means either an increase (stimulation) or a decrease (inhibition) in the expression of the gene encoding extracellular-signal-regulated kinase-6. In the context of the present invention, inhibition is the preferred form of modulation of gene expression and mRNA is a preferred target. In one embodiment of the present invention, the compounds of the present invention inhibit expression of extracellular-signal-regulated kinase-6 by at least 10% as measured in a suitable assay, such as those described in the examples below. In another embodiment, the compounds of the present invention inhibit expression of extracellular-signal-regulated kinase-6 by at least 25%. In still another embodiment of the invention, the compounds of the present invention inhibit expression of extracellular-signal-regulated kinase-6 by at least 40%. In yet a further embodiment of this invention, the compounds of the present invention inhibit expression of extracellular-signal-regulated kinase-6 by at least 50%. In a further embodiment of this invention, the compounds of the present invention inhibit expression of extracellular-signal-regulated kinase-6 by at least 60%. In another embodiment of this invention, the compounds of the present invention inhibit expression of extracellular-signal-regulated kinase-6 by at least 70% or at least 80% or higher as illustrated by the compounds identified in Examples 15, 21 and 22 below.

It is preferred to target specific nucleic acids for antisense. “Targeting” an antisense compound to a particular nucleic acid, in the context of this invention, is a multistep process. In this invention, the process begins with the identification of a nucleic acid sequence encoding the extracellular-signal-regulated kinase-6 for which the function is to be modulated. The targeting process also includes determination of a site or sites within this gene for the antisense interaction to occur such that the desired effect, e.g., detection or modulation of expression of the protein, results. Within the context of the present invention, one embodiment of an intragenic site for the extracellular-signal-regulated kinase-6 is the region encompassing the translation initiation or termination codon of the open reading frame (ORF) of the gene. Since, as is known in the art, the translation initiation codon is |typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding extracellular-signal-regulated kinase-6, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is another embodiment of a region of the extracellular-signal-regulated kinase-6 gene that may be targeted effectively. In another embodiment the 5′ untranslated region (5′UTR) of extracellular-signal-regulated kinase-6, known in the art to refer to the portion of the mRNA in the 5′ direction from the translation initiation codon, and thus including nucleotides between the 5′ cap site and the translation initiation codon of the mRNA or corresponding nucleotides on the gene, is the target region. In yet another embodiment, the 3′ untranslated region (3′UTR) extracellular-signal-regulated kinase-6, known in the art to refer to the portion of an mRNA in the 3′ direction from the translation termination codon, and thus including nucleotides between the translation termination codon and 3′ end of an mRNA or corresponding nucleotides on the gene is the target region. A further target region includes the 5′ cap of the mRNA for extracellular-signal-regulated kinase-6 that comprises an N7-methylated guanosine residue joined to the 5′-most residue of the mRNA via a 5′-5′ triphosphate linkage. The 5′ cap region of the mRNA is considered to include the 5′ cap structure itself as well as the first 50 nucleotides adjacent to the cap. In yet another embodiment, the 5′ cap region itself is also a target region according to this invention.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. In still other embodiments of this invention, mRNA splice sites, i.e., intron-exon junctions, are target regions of the gene encoding extracellular-signal-regulated kinase-6, and are particularly useful in situations where aberrant splicing is implicated in disease, or where an overproduction of a particular mRNA splice product is implicated in disease. In further embodiment, aberrant fusion junctions due to rearrangements or deletions are target regions. mRNA transcripts produced via the process of splicing of two (or more) mRNAs from different gene sources are known as “fusion transcripts”. Introns can be effective target regions for antisense compounds targeted, for example, to DNA or pre-mRNA of extracellular-signal-regulated kinase-6.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and extronic regions.

Upon excision of one or more exon or intron regions or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

Variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, oligonucleotides are chosen which are sufficiently complementary to the target, i.e., hybridize sufficiently well and with sufficient specificity, to give the desired effect. See, e.g., Tables 1-5 below. For example, Table 1 indicates oligonucleotides that hybridize to target regions of nucleotide 25 to 1631 of SEQ ID NO: 4. As another example, Table 3 indicates illustrative oligonucleotides that hybridize to target regions found within nucleotides 4 to 1728 of SEQ ID NO: 72; to regions within nucleotides 87 to 1542 of SEQ ID NO: 73; to regions within nucleotides 25-641 of SEQ ID NO: 74; to regions within nucleotides 455 to 836 of SEQ ID NO: 75; to regions within nucleotides 106 to 1223 of SEQ ID NO: 76 and to regions within nucleotides 495 and 514 of SEQ ID NO: 77.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable.

An antisense compound is specifically hybridizable when binding of the compound to the target DNA or RNA molecule interferes with the normal function of the target DNA or RNA to cause a loss of activity of extracellular-signal-regulated kinase-6, and there is a sufficient degree of complementarity to avoid non-specific binding of the antisense compound to non-target sequences under conditions in which specific binding is desired. Such conditions include, e.g., under physiological conditions in the case of in vivo assays or therapeutic treatment, and in the case of in vitro assays, under conditions in which the assays are performed. In one embodiment, the antisense compounds of the present invention comprise at least 80% sequence complementarity to a target region within the target nucleic acid of extracellular-signal-regulated kinase-6 to which they are targeted. In another embodiment, the antisense compounds of the present invention comprise at least 90% sequence complementarity to a target region within the target nucleic acid of extracellular-signal-regulated kinase-6 to which they are targeted. In still another embodiment of this invention, the antisense compounds of the present invention comprise at least 95% sequence complementarity to a target region within the target nucleic acid of extracellular-signal-regulated kinase-6 to which they are targeted. For example, an antisense compound in which 18 of 20 nucleobases of the antisense compound are complementary, and would therefore specifically hybridize, to a target region would represent 90 percent complementarity. Percent complementarity of an antisense compound with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656).

Antisense and other compounds of the invention, which hybridize to the target, extracellular-signal-regulated kinase-6, and inhibit expression of the target, are identified as taught herein. Representative sequences of these compounds are hereinbelow identified as embodiments of the invention. The sites to which these representative antisense compounds are specifically hybridizable are hereinbelow referred to as “illustrative target regions” and are therefore sites for targeting. As used herein the term “illustrative target region” is defined as at least an 8-nucleobase portion of a target region of extracellular-signal-regulated kinase-6, to which an active antisense compound is targeted. In another embodiment an illustrative target region is at least 15 nucleobases. In still another embodiment an illustrative target region is at least 20 nucleobases. In another embodiment an illustrative target region is at 30 nucleobases. In yet another embodiment an illustrative target region is at least 40 nucleobases. In still another embodiment an illustrative target region is at least 50 nucleobases. In another embodiment an illustrative target region is at 60 nucleobases. In still another embodiment an illustrative target region is at least 70 nucleobases. In another embodiment an illustrative target region is at least 80 nucleobases or more. In other embodiments, the illustrative target region consists of consecutive nucleobases. Thus, target regions 8-80 nucleobases in length comprising a stretch of at least eight (8) consecutive nucleobases selected from within the illustrative target regions are considered to be suitable target regions as well. While not wishing to be bound by theory, it is presently believed that these illustrative target regions represent regions of the target nucleic acid that are accessible for hybridization.

While the specific sequences of particular illustrative target regions are set forth below, one of skill in the art will recognize that these serve to illustrate and describe particular embodiments within the scope of the present invention. Additional target regions may be identified by one having ordinary skill using the teachings of this invention.

Exemplary additional target regions include DNA or RNA sequences that comprise at least the 8 consecutive nucleobases from the 5′-terminus of one of the illustrative target regions (the remaining nucleobases being a consecutive stretch of the same DNA or RNA beginning immediately upstream of the 5′-terminus of the target region and continuing until the DNA or RNA contains about 8 to about 80 nucleobases). Similarly additional target regions are represented by DNA or RNA sequences that comprise at least the 8 consecutive nucleobases from the 3′-terminus of one of the illustrative target regions (the remaining nucleobases being a consecutive stretch of the same DNA or RNA beginning immediately downstream of the 3′-terminus of the target region and continuing until the DNA or RNA contains about 8 to about 80 nucleobases). One having skill in the art, once armed with the teachings of the illustrative target regions described herein will be able, without undue experimentation, to identify further target regions. In addition, one having ordinary skill in the art using the teachings contained herein will also be able to identify additional compounds, including oligonucleotide probes and primers, that specifically hybridize to these illustrative target regions using techniques available to the ordinary practitioner in the art.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

For use in kits and diagnostics, the antisense compounds of the present invention, either alone or in combination with other antisense compounds or therapeutics, can be used as tools in differential and/or combinatorial analyses to elucidate expression patterns of a portion or the entire complement of extracellular-signal-regulated kinase-6 genes expressed within cells and tissues.

Expression patterns within cells or tissues treated with one or more antisense compounds are compared to control cells or tissues not treated with antisense compounds and the patterns produced are analyzed for differential levels of extracellular-signal-regulated kinase-6 gene expression as they pertain, for example, to disease association, signaling pathway, cellular localization, expression level, size, structure or function of the extracellular-signal-regulated kinase-6 genes examined. These analyses can be performed on stimulated or unstimulated cells and in the presence or absence of other compounds that affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U. S. A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytoometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in To, Comb. Chem. High Throughput Screen, 2000, 3, 235-41).

The specificity and sensitivity of antisense is also harnessed by those of skill in the art for therapeutic uses. Antisense oligonucleotides have been employed as therapeutic moieties in the treatment of disease states in animals, particularly mammals, and including humans. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below.

The antisense compounds in accordance with this invention preferably comprise compounds at least about 8 nucleobases in length (i.e. linked nucleosides). In one embodiment, antisense compounds of this invention are antisense oligonucleotides of at least about 12 nucleobases in length. In another embodiment, antisense compounds of this invention comprise about 20 nucleobases in length. In still another embodiment, antisense compounds of this invention comprise about 30 nucleobases in length. In yet another embodiment, antisense compounds of this invention comprise about 40 nucleobases in length. In still another embodiment, antisense compounds of this invention comprise about 50 nucleobases in length. In another embodiment, antisense compounds of this invention comprise about 60 nucleobases in length. In still another embodiment, antisense compounds of this invention comprise about 70 nucleobases in length. In yet another embodiment, antisense compounds of this invention comprise about 80 nucleobases in length. Antisense compounds include ribozymes, external guide sequence (EGS) oligonucleotides (oligozymes), and other short catalytic RNAs or catalytic oligonucleotides that hybridize to the target nucleic acid encoding extracellular-signal-regulated kinase-6 and modulate its expression.

Antisense compounds spanning from 8 to 80 nucleobases in length comprising a stretch of at least eight (8) consecutive nucleobases selected from within the illustrative antisense compounds described herein are considered to be suitable antisense compounds as well.

In one embodiment, exemplary antisense compounds include DNA or RNA sequences that comprise at least the 8 consecutive nucleobases from the 5′-terminus of one of the illustrative antisense compounds (the remaining nucleobases being a consecutive stretch of the same DNA or RNA beginning immediately upstream of the 5′-terminus of the antisense compound which is specifically hybridizable to the target nucleic acid and continuing until the DNA or RNA contains about 8 to about 80 nucleobases). Similarly, in another embodiment, such antisense compounds include at least 12 consecutive nucleobases from the 5′-terminus of one of the illustrative antisense compounds. In yet another embodiment, the antisense compound includes at least 20 consecutive nucleobases from the 5′-terminus of one of the illustrative antisense compounds. In a further embodiment, the antisense compound includes at least 30 consecutive nucleobases from the 5′-terminus of one of the illustrative antisense compounds. In yet another embodiment, the antisense compound includes at least 50 consecutive nucleobases from the 5′-terminus of one of the illustrative antisense compounds. In still another embodiment, the antisense compound includes at least 60 or more consecutive nucleobases from the 5′-terminus of one of the illustrative antisense compounds.

Similarly, in another embodiment antisense compounds are represented by DNA or RNA sequences that comprise at least the 8 consecutive nucleobases from the 3′-terminus of one of the illustrative antisense compounds (the remaining nucleobases being a consecutive stretch of the same DNA or RNA beginning immediately downstream of the 3′-terminus of the antisense compound which is specifically hybridizable to the target nucleic acid and continuing until the DNA or RNA contains about 8 to about 80 nucleobases). In another embodiment, such antisense compounds include at least 12 consecutive nucleobases from the 3′-terminus of one of the illustrative antisense compounds. In yet another embodiment, the antisense compound includes at least 20 consecutive nucleobases from the 3′-terminus of one of the illustrative antisense compounds. In a further embodiment, the antisense compound includes at least 30 consecutive nucleobases from the 3′-terminus of one of the illustrative antisense compounds. In yet another embodiment, the antisense compound includes at least So consecutive nucleobases from the 3′-terminus of one of the illustrative antisense compounds. In still another embodiment, the antisense compound includes at least 60 or more consecutive nucleobases from the 3′-terminus of one of the illustrative antisense compounds. One having skill in the art, once armed with the antisense compounds illustrated and other teachings herein will be able, without undue experimentation, to identify further antisense compounds of this invention.

Representative antisense and other compounds of the invention, which hybridize to the target, extracellular-signal-regulated kinase-6, and inhibit expression of the target, are identified below in Tables 1-5, as illustrative embodiments of the invention. While specific sequences of the antisense compounds are set forth herein, one of skill in the art will recognize that these serve to illustrate and describe particular embodiments within the scope of the present invention. Additional antisense compounds may be identified by one having ordinary skill using the teachings of this specification.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure, however, open linear structures are generally preferred. In addition, linear structures may also l have internal nucleobase complementarity and may therefore fold in a manner as to produce a double stranded structure. Within the oligonucleotide structure, the phosphate groups are commonly referred to as forming the internucleoside backbone of the oligonucleotide. The normal linkage or * backbone of RNA and DNA is a 3′ to 5′ phosphodiester linkage.

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue that may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— (known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5;519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in International Published Patent Application Nos. WO 98/39352 and WO 99/14226.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2 -amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b] [1,4]benzothiazin-2(3H)-one), G-clamps such as a substituted phenoxazine cytidine (e.g. 9-(2-aminoethoxy)-H-pyrimido[5,4-b] [1,4)benzoxazin-2(3H)-one), carbazole cytidine (2H-pyrimido [4,5-b) indol-2-one), pyridoindole cytidine (H-pyrido[3′,2′:4,Spyrrolo[2,3-dlpyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates that enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/0⁹196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chemn. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1, 2-di-O-hexadecyl-rac-glycero-3-H-phosphonate (manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmnacol. Exp. Ther., 1996, 277, 923-937). oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999), which is incorporated herein by reference in its entirety.

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to |confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. The cleavage of RNA:RNA hybrids can, in like fashion, be accomplished through the actions of endoribonucleases, such as interferon-induced RNAseL which cleaves both cellular and viral RNA. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described herein. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in International Published Patent Application No. WO 93/24510 to Gosselin et al., published Dec. 9, 1993 or in International Published Patent Application No. WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci. , 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits for a variety of conditions related to the expression of extracellular-signal-regulated kinase-6. Among such conditions are modulation of angiogenesis, i.e., the reduction or inhibition of new blood vessel growth. Such treatment is useful for the management of tumors, such as breast cancer tumors, the treatment of melanoma, among other diseases. For therapeutics, an animal, preferably a human, suspected of having a disease or disorder that can be treated by modulating the expression of extracellular-signal-regulated kinase-6 is treated by administering antisense compounds in accordance with this invention. The compounds of the invention can be utilized in pharmaceutical compositions by adding an effective amount of an antisense compound to a suitable pharmaceutically acceptable diluent or carrier. Us t e pheuantisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding extracellular-signal-regulated kinase-6, enabling sandwich and other assays to easily be constructed to exploit this fact. Hybridization of the antisense oligonucleotides of the invention with a nucleic acid encoding extracellular-signal-regulated kinase-6 can be detected by means known in the art. Such means may include conjugation of an enzyme to the oligonucleotide, radiolabelling of the oligonucleotide or any other suitable detection means. Kits using such detection means for detecting the level of extracellular-signal-regulated kinase-6 in a sample may also be prepared.

The present invention also includes pharmaceutical compositions and formulations that include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. applications Ser. Nos. 08/886,829 (filed Jul. 1, 1997), 09/108,673 (filed Jul. 1, 1998), 09/256,515 (filed Feb. 23, 1999), 09/082,624 (filed May 21, 1998) and 09/315,298 (filed May 20, 1999), each of which is incorporated herein by reference in their entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The compositions of the present invention may be prepared and formulated as emulsions. Emulsions are typically heterogenous systems of one liquid dispersed in another in the form of droplets usually exceeding 0.1 μm in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, 1Marcel Dekker, Inc., New York, N.Y., volume 1, p. 199).

Synthetic surfactants, also known as surface active agents, have found wide applicability in the formulation:of emulsions and have been reviewed in the literature (Rieger, in Pharmaceutical Dosage Forms, Lieberman, Rieger and Banker (Eds.), 1988:, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different |classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, |p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile, which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the -standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to-one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides. microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome that is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc.,: New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposdmes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes that interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 98:0-985).

Liposomes that are pH-sensitive or negatively-charged entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharmna. Sci., 1994, 4, 6, 466).

Liposomes also include “sterically stabilized” liposomes. This term, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and International Published Patent Application No. WO 88/04924, both to Allen et al., refer to liposomes comprising (1) sphingomyelin and (2) the ganglioside G_{M1} or a galactocerebroside sulfate ester. U.S. Pat. No. 5,543,152 (Webb et al.) refers to liposomes comprising sphingomyelin. Liposomes comprising 1,2-sn-dimyristoylphosphatidylcholine are disclosed in International Published Patent Application No. WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, which contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and International Published Patent Application No. WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in International Published Patent Application No. WO 91/05545 and U.S. Pat. No. 5,225,212 (both to Martin et al.) and in International Published Patent Application No. WO 94/20073 (Zalipsky et al.) Liposomes comprising PEG-modified ceramide lipids are described in International Published Patent Application No. WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. International Published Patent Application No. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. International Published Patent Application No. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets that are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB) The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to enable the efficient delivery of nucleic acids, particularly oligonucleotides, to the skin of animals. Most drugs are, present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., -1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For, example, cationic lipids, such as lipofectin (Junichi et al, U.S. Pat. No. 5,705,188), cationic glycerol derivatives, and polycationic molecules, such as polylysine (Lollo et al., International Published Patent Application No. WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration that do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxvmethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine,: hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same.

### EXAMPLES

### Example 1

### Nucleoside Phosphoramidites for Oligonucleotide Synthesis Deoxy and 2′-Alkoxy Amidites

2′-Deoxy and 2′-methoxy beta-cyanoethyldiisopropyl phosphoramidites were purchased from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, optimized synthesis cycles were developed that incorporate multiple steps coupling longer wait times relative to standard synthesis cycles.

The following abbreviations are used in the text: thin layer chromatography (TLC), melting point (MP), high pressure liquid chromatography (HPLC), Nuclear Magnetic Resonance (NMR), argon (Ar), methanol (MeOH), dichloromethane (CH₂Cl₂), triethylamine (TEA), dimethyl formamide (DMF), ethyl acetate (EtOAc), dimethyl sulfoxide (DMSO), tetrahydrofuran (THF).

Oligonucleotides containing 5-methyl-2′-deoxycytidine (5-Me-dC) nucleotides were synthesized according to published methods (Sanghvi, et. al., Nucleic Acids Research, 1993, 21, 3197-3203) using commercially available phosphoramidites (Glen Research, Sterling Va. or ChemGenes, Needham Mass.) or prepared as follows: =cl Preparation of 5′-O-Dimethoxytrityl-thymidine intermediate for 5-methyl dC amidite

To a 50 L glass reactor equipped with air stirrer and Ar gas line was added thymidine (1.00 kg, 4.13 mol) in anhydrous pyridine (6′ L) at ambient temperature. Dimethoxytrityl (DMT) chloride (1.47 kg, 4.34 mol, 1.05 eq) was added as a solid in four portions over 1 h. After 30 min, TLC indicated approx. 95% product, 2% thymidine, 5% DMT reagent and by-products and 2% 3′,5′-bis DMT product (Rf in EtOAc 0.45, 0.05, 0.98, 0.95 respectively). Saturated sodium bicarbonate (4 L) and CH₂Cl2 were added with stirring (pH of the aqueous layer 7.5). An additional 18 L of water was added, the mixture was stirred, the phases were separated, and the organic layer was transferred to a second 50 L vessel. The aqueous layer was extracted with additional CH₂Cl₂ (2×2 L). The combined organic layer was washed with water (10 L) and then concentrated in a rotary evaporator to approx. 3.6 kg total weight. This was redissolved in CH₂Cl₂ (3.5 L), added to the reactor followed by water (6 L) and hexanes (13 L). The mixture was vigorously stirred and seeded to give a fine white suspended solid starting at the interface. After stirring for 1 h, the suspension was removed by suction through a ½″ diameter teflon tube into a 20 L suction flask, poured onto a 25 cm Coors Buchner funnel, washed with water (2×3 L) and a mixture of hexanes-CH₂Cl₂ (4:1, 2×3 L) and allowed to air dry overnight in pans (1″ deep). This was further dried in a vacuum oven (75° C., 0.1 mm Hg, 48 h) to a constant weight of 2072 g (93%) of a white solid, (mp 122-124° C.). TLC indicated a trace contamination of the bis DMT product. NMR spectroscopy also indicated that 1-2 mole percent pyridine and about 5 mole percent of hexanes was still present.

### Preparation of 5′-O-Dimethoxytrityl-2′-deoxy-5-methylcytidine intermediate for 5-methyl-dC amidite

To a 50 L Schott glass-lined steel reactor equipped with an electric stirrer, reagent addition pump (connected to an addition funnel), heating/cooling system, internal thermometer and an Ar gas line was added 5′-O-dimethoxytrityl-thymidine (3.00 kg, 5.51 mol), anhydrous acetonitrile (25 L) and TEA (12.3 L, 88.4 mol, 16 eq). The mixture was chilled with stirring to −10° C. internal temperature (external −20° C.). Trimethylsilylchloride (2.1 L, 16.5 mol, 3.0 eq) was added over 30 minutes while maintaining the internal temperature below −5° C., followed by a wash of anhydrous acetonitrile (1 L). Note: the reaction is mildly exothermic and copious hydrochloric acid fumes form over the course of the addition. The reaction was allowed to warm to 0° C. and the reaction progress was confirmed by TLC (EtOAc-hexanes 4:1; R_{f} 0.43 to 0.84 of starting material and silyl product, respectively). Upon completion, triazole (3.05 kg, 5 44 mol, 8.0 eq) was added the reaction was cooled to −20° C. internal temperature (external −30° C.). Phosphorous oxychloride (1035 mL, 11.1 mol, 2.01 eq) was added over 60 min so as to maintain the temperature between −20° C. and −10° C. during the strongly exothermic process, followed by a wash of anhydrous acetonitrile (1 L). The reaction was warmed to 0° C. and stirred for 1 h. TLC indicated a complete conversion to the triazole product (R_{f} 0.83 to 0.34 with the product spot glowing in long wavelength UV light). The reaction mixture was a peach-colored thick suspension, which turned darker red upon warming without apparent decomposition. The reaction was cooled to −15° C. internal temperature and water (5 L) was slowly added at a rate to maintain the temperature below +10° C. in order to quench the reaction and to form a homogenous solution. (Caution: this reaction is initially very strongly exothermic). Approximately one-half of the reaction volume (22 L) was transferred by air pump to another vessel, diluted with EtOAc (12 L) and extracted with water (2×8 L). The combined water layers were back-extracted with EtOAc (6 L). The water layer was discarded and the organic layers were concentrated in a 20 L rotary evaporator to an oily foam. The foam was coevaporated with anhydrous acetonitrile (4 L) to remove EtOAc. (note: dioxane may be used instead of anhydrous acetonitrile if dried to a hard foam). The second half of the reaction was treated in the same way. Each residue was dissolved in dioxane (3 L) and concentrated ammonium hydroxide (750 mL) was added. A homogenous solution formed in a few minutes and the reaction was allowed to stand overnight (although the reaction is complete within 1 h).

TLC indicated a complete reaction (product R_{f} 0.35 in EtOAc-MeOH 4:1). The reaction solution was concentrated on a rotary evaporator to a dense foam. Each foam was slowly redissolved in warm EtOAc (4 L; 50° C.), combined in a 50 L glass reactor vessel, and extracted with water (2×4L) to remove the triazole by-product. The water was back-extracted with EtOAc (2 L). The organic layers were combined and concentrated to about 8 kg total weight, cooled to 0° C. and seeded with crystalline product. After 24 hours, the first crop was collected on a 25 cm Coors Buchner funnel and washed repeatedly with EtOAc (3×3L) until a white powder was left and then washed with ethyl ether (2×3L). The solid was put in pans (1″ deep) and allowed to air dry overnight. The filtrate was concentrated to an oil, then redissolved in EtOAc (2 L), cooled and seeded as before. The second crop was collected and washed as before (with proportional solvents) and the filtrate was first extracted with water (2×1 L) and then concentrated to an oil. The residue was dissolved in EtOAc (1 L) and yielded a third crop which was treated as above except that more washing was required to remove a yellow oily layer.

After air-drying, the three crops were dried in a vacuum oven (50° C., 0.1 mm Hg, 24 h) to a constant weight (1750, 600 and 200 g, respectively) and combined to afford 2550 g (85%) of a white crystalline product (MP 215-217° C.) when TLC and NMR spectroscopy indicated purity. The mother liquor still contained mostly product (as determined by TLC) and a small amount of triazole (as determined by NMR spectroscopy), bis DMT product and unidentified minor impurities. If desired, the mother liquor can be purified by silica gel chromatography using a gradient of MeOH (0-25%) in EtOAc to further increase the yield.

### Preparation of 5′-O-Dimethoxytrityl-2′-deoxy-N4-benzoyl-5-methylcytidine penultimate intermediate for 5-methyl dC amidite

Crystalline 5′-O-dimethoxytrityl-5-methyl-2′-deoxycytidine (2000 g, 3.68 mol) was dissolved in anhydrous DMF (6.0 kg) at ambient temperature in a 50 L glass reactor vessel equipped with an air stirrer and argon line. Benzoic anhydride (Chem Impex not Aldrich, 874 g, 3.86 mol, 1.05 eq) was added and the reaction was stirred at ambient temperature for 8 h. TLC (CH₂Cl₂-EtOAc; CH₂Cl₂-EtOAc 4:1; R_{f} 0.25) indicated approx. 92% complete reaction. An additional amount of benzoic anhydride (44 g, 0.19 mol) was added. After a total of 18 h, TLC indicated approx. 96% reaction completion. The solution was diluted with EtOAc (20 L), TEA (1020 mL, 7.36 mol, ca 2.0 eq) was added with stirring, and the mixture was extracted with water (15 L, then 2×10 L). The aqueous layer was removed (no back-extraction was needed) and the organic layer was concentrated in 2×20 L rotary evaporator flasks until a foam began to form. The residues were coevaporated with acetonitrile (1.5 L each) and dried (0.1 mm Hg, 25° C., 24 h) to 2520 g of a dense foam. High pressure liquid chromatography (HPLC) revealed a contamination of 6.3% of N4, 3′-O-dibenzoyl product, but very little other impurities.

The product was purified by Biotage column chromatography (5 kg Biotage) prepared with 65:35:1 hexanes-EtOAc-TEA (4 L). The crude product (800 g), dissolved in CH₂Cl₂ (2 L), was applied to the column. The column was washed with the 65:35:1 solvent mixture (20 kg), then 20:80:1 solvent mixture (10 kg), then 99:1 EtOAc:TEA (17 kg). The fractions containing the product were collected, and any fractions containing the product and impurities were retained and resubjected to column chromatography. The column was re-equilibrated with the original 65:35:1 solvent mixture (17 kg). A second batch of crude product (840 g) was applied to the column as before. The column was washed with the following solvent gradients: 65:35:1 (9 kg), 55:45:1 (20 kg), 20:80:1 (10 kg), and 99:1 EtOAc:TEA(15 kg). The column was reequilibrated as above, and a third batch of the crude product (850 g) plus impure fractions recycled from the two previous columns (28 g) was purified following the procedure for the second batch. The fractions containing pure product combined and concentrated on a 20 L rotary evaporator, co-evaporated with acetontirile (3 L) and dried (0.1 mm Hg, 48 h, 25° C.) to a constant weight of 2023 g (85%) of white foam and 20 g of slightly contaminated product from the third run. HPLC indicated a purity of 99.8% with the balance as the diBenzoyl product.

### [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite)

5-O-(4,4′-Dimethoxytriphenylmethyl)-2′-deoxy-N⁴-benzoyl-5-methylcytidine (998 g, 1.5 mol) was dissolved in anhydrous DMF (2 L). The solution was co-evaporated with is toluene (300 ml) at 50° C. under reduced pressure, then cooled to room temperature and 2-cyanoethyl tetraisopropylphosphorodiamidite (680 g, 2.26 mol) and tetrazole (52.5 g, 0.75 mol) were added. The mixture was shaken until all tetrazole was dissolved, N-methylimidazole (15 ml) was added and the mixture was left at room temperature for S hours. TEA (300 ml) was added; the mixture was diluted with DMF (2.5 L) and water (600 ml), and extracted with hexane (3×3 L). The mixture was diluted with water (1.2 L) and extracted with a mixture of toluene (7.5 L) and hexane (6 L). The two layers were separated, the upper layer was washed with DMF-water (7:3 v/v, 3×2 L) and water (3×2 L), and the phases were separated. The organic layer was dried (Na₂SO₄), filtered and rotary evaporated. The residue was co-evaporated with acetonitrile (2×2 L) under reduced pressure and dried to a constant weight (25° C., 0.1 mm Hg, 40 h) to afford 1250 g an off-white foam solid (96%).

### 2′-Fluoro amidites

### 2′-Fluorodeoxyadenosine amidites

2′-fluoro oligonucleotides were synthesized as described previously [Kawasaki, et. al., J. Med. Chem., 1993, 36, 831-841] and U.S. Pat. No. 5,670,633, herein incorporated by reference. The preparation of 2′-fluoropyrimidines containing a 5-methyl substitution are described in U.S. Pat. No. 5,861,493. Briefly, the protected nucleoside N6-benzoyl-2′-deoxy-2′-fluoroadenosine was synthesized utilizing commercially available 9-beta-D-arabinofuranosyladenine as starting material and whereby the 2′-alpha-fluoro atom is introduced by a S_{N}2-displacement of a 2′-beta-triflate group. Thus N6-benzoyl-9-beta-D-arabinofuranosyladenine was selectively protected in moderate yield as the 3′,5′-ditetrahydropyranyl (THP) intermediate. Deprotection of the THP and N6-benzoyl groups was accomplished using standard methodologies to obtain the 5′-dimethoxytrityl-(DMT) and 5′-DMT-3′-phosphoramidite intermediates.

### 2′-Fluorodeoxyguanosine

The synthesis of 2′-deoxy-2′-fluoroguanosine was accomplished using tetraisopropyldisiloxanyl (TPDS) protected 9-beta-D-arabinofuranosylguanine as starting material, and conversion to the intermediate isobutyryl-arabinofuranosylguanosine. Alternatively, isobutyryl-arabinofuranosylguanosine was prepared as described by Ross et al., (Nucleosides & Nucleosides, 16, 1645, 1997). Deprotection of the TPDS group was followed by protection of the hydroxyl group with THP to give isobutyryl di-THP protected arabinofuranosylguanine. Selective O-deacylation and triflation was followed by treatment of the crude product with fluoride, then deprotection of the THP groups. Standard methodologies were used to obtain the 5′-DMT- and 5′-DMT-3′-phosphoramidites.

### 2′-Fluorouridine

Synthesis of 2′-deoxy-2′-fluorouridine was accomplished by the modification of a literature procedure in which 2,2′-anhydro-1-beta-D-arabinofuranosyluracil was treated with 70% hydrogen fluoride-pyridine. Standard procedures were used to obtain the 5′-DMT and 5′-DMT-3′phosphoramidites.

### 2′-Fluorodeoxycytidine

2′-deoxy-2′-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′phosphoramidites.

### 2′-O-(2-Methoxyethyl) modified amidites

2′-O-Methoxyethyl-substituted nucleoside amidites (otherwise known as MOE amidites) are prepared as follows, or alternatively, as per the methods of Martin, P., (Helvetica Chimica Acta, 1995, 78, 486-504)

### Preparation of 2′-O-(2-methoxyethyl)-5-methyluridine intermediate

2,2′-Anhydro-5-methyl-uridine (2000 g, 8.32 mol), tris(2-methoxyethyl)borate (2504 g, 10.60 mol), sodium bicarbonate (60 9, 0.70 mol) and anhydrous 2-methoxyethanol (5 L) were combined in a 12 L three necked flask and heated to 130° C. (internal temp) at atmospheric pressure, under an argon atmosphere with stirring for 21 h. TLC indicated a complete reaction. The solvent was removed under reduced pressure until a sticky gum formed (50-85° C. bath temp and 100-11 mm Hg) and the residue was redissolved in water (3 L) and heated to boiling for 30 min in order the hydrolyze the borate esters. The water was removed under reduced pressure until a foam began to form and then the process was repeated. HPLC indicated about 77% product, 15% dimer (5′ of product attached to 2′ of starting material) and unknown derivatives, and the balance was a single unresolved early eluting peak.

The gum was redissolved in brine (3 L), and the flask was rinsed with additional brine (3 L). The combined aqueous solutions were extracted with chloroform (20 L) in a heavier-than continuous extractor for 70 h. The chloroform layer was concentrated by rotary evaporation in a 20 L flask to a sticky foam (2400 g). This was coevaporated with MeOH (400 mL) and EtOAc (8 L) at 75° C. and 0.65 atm until the foam dissolved at which point the vacuum was lowered to about 0.5 atm. After 2.5 L of distillate was collected a precipitate began to form and the flask was removed from the rotary evaporator and stirred until the suspension reached ambient temperature. EtOAc (2 L) was added and the slurry was filtered on a 25 cm table top Buchner funnel and the product was washed with EtOAc (3×2 L). The bright white solid was air dried in pans for 24 h then further dried in a vacuum oven (50° C., 0.1 mm Hg, 24 h) to afford 1649 g of a white crystalline solid (mp 115.5-116.5° C.).

The brine layer in the 20 L continuous extractor was further extracted for 72 h with recycled chloroform. The chloroform was concentrated to 120 g of oil and this was combined with the mother liquor from the above filtration (225 g), dissolved in brine (250 mL) and extracted once with chloroform (250 mL). The brine solution was continuously extracted and the product was crystallized as described above to afford an additional 178 g of crystalline product containing about 2% of thymine. The combined yield was 1827 g (69.4%). HPLC indicated about 99.5% purity with the balance being the dimer.

### Preparation of 5′-O-DMT-2′-O-(2-methoxyethyl)-5-methyluridine penultimate intermediate

In a 50:L glass-lined steel reactor, 2′-O-(2-methoxyethyl)-5-methyl-uridine (MOE-T, 1500 g, 4.738 mol), lutidine (1015 g, 9.476 mol) were dissolved in anhydrous acetonitrile (15 L). The solution was stirred rapidly and chilled to −10° C. (internal temperature). Dimethoxytriphenylmethyl chloride (1765.7 g, 5.21 mol) was added as a solid in one portion. The reaction was allowed to warm to −2° C. over 1 h. (Note: The reaction was monitored closely by TLC (EtOAc) to determine when to stop the reaction so as to not generate the undesired bis-DMT substituted side product). The reaction was allowed to warm from −2 to 3° C. over 25 min. then quenched by adding MeOH (300 mL) followed after 10 min by toluene (16 L) and water (16 L). The |solution was transferred to a clear 50 L vessel with a bottom outlet, vigorously stirred for 1 minute, and the layers separated. The aqueous layer was removed and the organic layer was washed successively with 10% aqueous citric acid (8 L) and water (12 L). The product was then extracted into the aqueous phase by washing the toluene solution with aqueous sodium hydroxide (0.5N, 16 L and 8 L). The combined aqueous layer was overlayed with toluene (12 L) and solid citric acid (8 moles, 1270 g) was added with vigorous stirring to lower the pH of the aqueous layer to 5.5 and extract the product into the toluene. The organic layer was washed with water (10 L) and TLC of the organic layer indicated a trace of DMT-O-Me, bis DMT and dimer DMT.

The toluene solution was applied to a silica gel column (6 L sintered glass funnel containing approx. 2 kg of silica gel slurried with toluene (2 L) and TEA(25 mL)) and the fractions were eluted with toluene (12 L) and EtOAc (3×4 L) using vacuum applied to a filter flask placed below the column. The first EtOAc fraction containing both the desired product and impurities were resubjected to column chromatography as above. The clean fractions were combined, rotary evaporated to a foam, coevaporated with acetonitrile (6 L) and dried in a vacuum oven (0.1 mm Hg, 40 h, 40° C.) to afford 2850 g of a white crisp foam. NMR spectroscopy indicated a 0.25 mole % remainder of acetonitrile (calculates to be approx. 47 g) to give a true dry weight of 2803 g (96%). HPLC indicated that the product was 99.41% pure, with the remainder being 0.06 DMT-O-Me, 0.10 unknown, 0.44 bis DMT, and no detectable dimer DMT or 3′-O-DMT.

### Preparation of [5′-O -(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite)

5′-0-(4,4′-Dimethoxytriphenylmethyl)-2′-O-(2-methoxyethyl)-5-methyluridine (1237 g, 2.0 mol) was dissolved in anhydrous DMF (2.5 L). The solution was co-evaporated with toluene (200 ml) at 50° C. under reduced pressure, then cooled to room temperature and 2-cyanoethyl tetraisopropylphosphorodiamidite (900 g, 3.0 mol) and tetrazole (70 g, 1.0 mol) were added. The mixture was shaken until all tetrazole was dissolved, N-methylimidazole (20 ml) was added and the solution was left at room temperature for 5 hours. TEA (200 ml) was added, the mixture was diluted with DMF (3.5 L) and water (600 ml) and extracted with hexane (3×3 L). The mixture was diluted with water (1.6 L) and extracted with the mixture of toluene (12 L) and hexanes (9 L). The upper layer was washed with DMF-water (7:3 v/v, 3×3 L) and water (3×L). The organic layer was dried (Na₂SO₄), filtered and evaporated. The residue was co-evaporated with acetonitrile (2×2 L) under reduced pressure and dried in a vacuum oven (25° C., 0.1 mm Hg, 40 h) to afford 1526 g of an off-white foamy solid (95%).

### Preparation of 5′-O-Dimethoxytrityl-2′-O-(2-methoxyethyl)-5-ethylcytidine intermediate

To a 50 L Schott glass-lined steel reactor equipped with an electric stirrer, reagent addition pump (connected to an addition funnel), heating/cooling system, internal thermometer and argon gas line was added 5′-O-dimethoxytrityl-2′-O-(2-methoxyethyl)-5-methyl-uridine (2.616 kg, 4.23 mol, purified by base extraction only and no scrub column), anhydrous acetonitrile (20 L), and TEA (9.5 L, 67.7 mol, 16 eq). The mixture was chilled with stirring to −10° C. internal temperature (external −20° C.). Trimethylsilylchloride (1.60 L, 12.7 mol, 3.0 eq) was added over 30 min. while maintaining the internal temperature below −5° C., followed by a wash of anhydrous acetonitrile (1 L) (Note: the reaction is mildly exothermic and copious hydrochloric acid fumes form over the course of the addition). The reaction was allowed to warm to 0° C. and the reaction progress was confirmed by TLC (EtOAc, R_{f} 0.68 and 0.87 for starting material and silyl product, respectively). Upon completion, triazole (2.34 kg, 33.8 mol, 8.0 eq) was added the reaction was cooled to −20° C. internal temperature (external −30° C.). Phosphorous oxychloride (793 mL, 8.51 mol, 2.01 eq) was added slowly over 60 min so as to maintain the temperature between −20° C. and −10° C. (note: strongly exothermic), followed by a wash of anhydrous acetonitrile (1 L). The reaction was warmed to 0° C. and stirred for 1 h, at which point it was an off-white thick suspension. TLC indicated a complete conversion to the triazole product (EtOAc, R_{f} 0.87 to 0.75 with the product spot glowing in long wavelength UV light). The reaction was cooled to −15° C. and water (5 L) was slowly added at a rate to maintain the temperature below +10° C. in order to quench the reaction and to form a homogenous solution. (Caution: this reaction is initially very strongly exothermic). Approximately one-half of the reaction volume (22 L) was transferred by air pump to another vessel, diluted with EtOAc (12 L) and extracted with water (2×8 L). The second half of the reaction was treated in the same way. The combined aqueous layers were back-extracted with EtOAc (8 L) The organic layers were combined and concentrated in a 20 L rotary evaporator to an oily foam. The foam was coevaporated with anhydrous acetonitrile (4 L) to remove EtOAc. (Note: dioxane may be used instead of anhydrous acetonitrile if dried to a hard foam). The residue was dissolved in dioxane (2 L) and concentrated ammonium hydroxide (750 mL) was added. A homogenous solution formed in a few minutes and the reaction was allowed to stand overnight

TLC indicated a complete reaction (CH₂Cl₂-acetone-MeOH, 20:5:3, R_{f} 0.51). The reaction solution was concentrated on a rotary evaporator to a dense foam and slowly redissolved in warm CH₂Cl₂ (4 L, 40° C.) and transferred to a 20 L glass extraction vessel equipped with a air-powered stirrer. The organic layer was extracted with water (2×6 L) to remove the triazole by-product. (Note: In the first extraction an emulsion formed which took about 2 h to resolve). The water layer was back-extracted with CH₂Cl₂ (2×2 L), which in turn was washed with water (3 L). The combined organic layer was concentrated in 2×20 L flasks to a gum and then recrystallized from EtOAc seeded with crystalline product. After sitting overnight, the first crop was collected on a 25 cm Coors Buchner funnel and washed repeatedly with EtOAc until a white free-flowing powder was left (about 3×3 L). The filtrate was concentrated to an oil recrystallized from EtOAc, and collected as above. The solid was air-dried in pans for 48 h, then further dried in a vacuum oven (50° C., 0.1 mm Hg, 17 h) to afford 2248 g of a bright white, dense solid (86%). An HPLC analysis indicated both crops to be 99.4% pure and NMR spectroscopy indicated only a faint trace of EtOAc remained.

### Preparation of 5′-O-dimethoxytrityl-2′-O-(2-methoxyethyl)-N4-benzoyl-5-methyl-cytidine penultimate intermediate:

Crystalline 5′-O-dimethoxytrityl-2′-O-(2-methoxyethyl)-5-methyl-cytidine (1000 g, 1.62 mol) was suspended in anhydrous DMF (3 kg) at ambient temperature and stirred under an Ar atmosphere. Benzoic anhydride (439.3 g, 1.94 mol) was added in one portion. The solution clarified after 5 hours and was stirred for 16 h. HPLC indicated 0.45% starting material remained (as well as 0.32% N4, 3′-O-bis Benzoyl) An additional amount of benzoic anhydride (6.0 g, 0.0265 mol) was added and after 17 h, HPLC indicated-no starting material was present. TEA (450 mL, 3.24 mol) and toluene (6 L) were added with stirring for 1 minute. The solution was washed with water (4×4 L), and brine (2×4 L). The organic layer was partially evaporated on a 20 L rotary evaporator to remove 4 L of toluene and traces of water. HPLC indicated that the bis benzoyl side product was present as a 6% impurity. The residue was diluted with toluene (7 L) and anhydrous DMSO (200 mL, 2.82 mol) and sodium hydride (60% in oil, 70 g, 1.75 mol) was added in one portion with stirring at ambient temperature over 1 h. The reaction was quenched by slowly adding then washing with aqueous citric acid (10%, 100 mL over 10 min, then 2×4 L), followed by aqueous sodium bicarbonate (2%, 2 L), water (2×4 L) and brine (4 L). The organic layer was concentrated on a 20 L rotary evaporator to about 2 L total volume. The residue was purified by silica gel column chromatography (6 L Buchner funnel containing 1.5 kg of silica gel wetted with a solution of EtOAc-hexanes-TEA(70:29:1)). The product was eluted with the same solvent (30 L) followed by straight EtOAc (6 L). The fractions containing the product were combined, concentrated on a rotary evaporator to a foam and then dried in a vacuum oven (50° C., 0.2 mm Hg, 8 h) to afford 1155 g of a crisp, white foam (98%). HPLC indicated a purity of >99.7%.

### Preparation of [5′-0-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite)

5′-O-(4,4′-Dimethoxytriphenylmethyl)-2′-O-(2-methoxyethyl)-N⁴-benzoyl-5-methylcytidine (1082 g, 1.5 mol) was dissolved in anhydrous DMF (2 L) and co-evaporated with toluene (300 ml) at 50 ° C. under reduced pressure. The mixture as cooled to room temperature and 2-cyanoethyl etraisopropylphdsphorodiamidite (680 g, 2.26 mol) and etrazole (52.5 g, 0.75 mol) were added. The mixture was haken until all tetrazole was dissolved, N-methylimidazole (30 ml) was added, and the mixture was left at room temperature for 5 hours. TEA (300 ml) was added, the mixture as diluted with DMF (1 L) and water (400 ml) and extracted with hexane (3×3 L). The mixture was diluted with water (1.2 L) and extracted with a mixture of toluene (9 L) and hexanes (6 L). The two layers were separated and the upper layer was washed with DMF-water (60:40 v/v, 3×3 L) and water (3×2 L). The organic layer was dried (Na₂SO₄), filtered and evaporated. The residue was co-evaporated with acetonitrile (2×2 L) under reduced pressure and dried in a vacuum oven (25° C., 0.1 mm Hg, 40 h) to afford 1336 9 of an off-white foam (97%).

### Preparation of [5′-O -(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amidite)

5′-O-(4,4′-Dimethoxytriphenylmethyl)-2′-O-(2-methoxyethyl)-N⁶-benzoyladenosine (purchased from Reliable Biopharmaceutical, St. Louis, Mo.), 1098 g, 1.5 mol) was dissolved in anhydrous DMF (3 L) and co-evaporated with toluene (300 ml) at 50° C. The mixture was cooled to room temperature and 2-cyanoethyl tetraisopropylphosphorodiamidite (680 g, 2.26 mol) and tetrazole (78.8 g, 1.24 mol) were added. The mixture was shaken until all tetrazole was dissolved, N-methylimidazole (30 ml) was added, and mixture was left at room temperature for S hours. TEA (300 ml) was added, the mixture was diluted with DMF (1 L) and water (400 ml) and extracted with hexanes (3×3 L). The mixture was diluted with water (1.4 L) and extracted with the mixture of toluene (9 L) and hexanes (6 L). The two layers were separated and the upper layer was washed with DMF-water (60:40, v/v, 3×3 L) and water (3×2 L). The organic layer was dried (Na₂SO₄), filtered and evaporated to a sticky foam. The residue was co-evaporated with acetonitrile (2.5 L) under reduced pressure and dried in a vacuum oven (25 ° C., 0.1 mm Hg, 40 h) to afford 1350 g of an off-white foam solid (96%).

### Preparation of [5′-O-(4,4′-Dimethoxytriphenylmethyl)-2′-O-(2-methoxyethyl)-N4 -isobutyrylguanosin-3′-O-yl]-2-cyanoethyl-N,N-diisopropylphosphoramidite (MOE G amidite)

5′-O-(4,4′-Dimethoxytriphenylmethyl)-2′-O-(2-methoxyethyl)-N⁴-isobutyrlguanosine (purchased from Reliable Biopharmaceutical, St. Louis, Mo., 1426 g, 2.0 mol) was dissolved in anhydrous DMF (2 L). The solution was co-evaporated with toluene (200 ml) at 50 ° C., cooled to room temperature and 2-cyanoethyl tetraisopropylphosphorodiamidite (900 g, 3.0 mol) and tetrazole (68 g, 0.97 mol) were added. The mixture was shaken until all tetrazole was dissolved, N-methylimidazole (30 ml) was added, and the mixture was left at room temperature for 5 hours. TEA (300 ml) was added, the mixture was diluted with DMF (2 L) and water (600 ml) and extracted with hexanes (3×3 L). The mixture was diluted with water (2 L) and extracted with a mixture of toluene (10 L) and hexanes (5 L). The two layers were separated and the upper layer was washed with DMF-water (60:40,. v/v, 3×3 L). EtOAc (4 L) was added and the solution was washed with water (3×4 L). The organic layer was dried (Na₂SO₄), filtered and evaporated to approx. 4 kg. Hexane (4 L) was added, the mixture was shaken for 10 min, and the supernatant liquid was decanted. The residue was co-evaporated with acetonitrile (2×2 L) under reduced pressure and dried in a vacuum oven (25° C., 0.1 mm Hg, 40 h) to afford 1660 g of an off-white foamy solid (91%).

### 2′-O-(Aminooxyethyl) nucleoside amidites and 2′-O-(dimethylaminooxyethyl) nucleoside amidites

### 2′-(Dimethylaminooxyethoxy) nucleoside amidites

2′-(Dimethylaminooxyethoxy) nucleoside amidites (also known in the art as 2′-O-(dimethylaminooxyethyl) nucleoside amidites) are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

### 5′-O-tert-Butyldiphenylsilyl-O²-2′-anhydro-5-methyluridine

O²-2′-anhydro-5-methyluridine (Pro. Bio. Sint., Varese, Italy, 100.0 g, 0.416 mmol), dimethylatninopyridine (0.66 g, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, 1.1 eq, 0.458 mmol) was added in one portion. The reaction was stirred for 16 h at ambient temperature. TLC (R_{f} 0.22, EtOAc) indicated a complete reaction. The solution was concentrated under reduced pressure to a thick oil. This was partitioned between CH₂Cl₂ (1 L) and saturated sodium bicarbonate (2×1 L) and brine (1 L). The organic layer was dried over sodium sulfate, filtered, and concentrated under reduced pressure to a thick oil. The oil was dissolved in a 1:1 mixture of EtOAc and ethyl ether (600 mL) and cooling the solution to −10° C. afforded a white crystalline solid which was collected by filtration, washed with ethyl ether (3×2 00 mL) and dried (40° C., 1 mm Hg, 24 h) to afford 149 g of white solid (74.8%). TLC and NMR spectroscopy were consistent with pure product.

### 5′-O-tert-Butyldiphenylsilyl-2′-O-(2-hydroxyethyl)-5-methyluridine

In the fume hood, ethylene glycol (350 mL, excess) was added cautiously with manual stirring to a 2 L stainless steel pressure reactor containing borane in tetrahydrofuran (1.0 M, 2.0 eq, 622 mL). (Caution: evolves hydrogen gas). 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure<100 psig). The reaction vessel was cooled to ambient temperature and opened. TLC (EtOAc, R_{f} 0.67 for desired product and R_{f} 0.82 for ara-T side product) indicated about 70% conversion to the product. The solution was concentrated under reduced pressure (10 to 1 mm Hg) in a warm water bath (40-100° C.) with the more extreme conditions used to remove the ethylene glycol. (Alternatively, once the THF has evaporated the solution can be diluted with water and the product extracted into EtOAc). The residue was purified by column chromatography (2 kg silica gel, EtOAc-hexanes gradient 1:1 to 4:1). The appropriate fractions were combined, evaporated and dried to afford 84 g of a white crisp foam (50%), contaminated starting material (17.4 g, 12% recovery) and pure reusable starting material (20 g, 13% recovery). TLC and NMR spectroscopy were consistent with 99% pure product.

### 2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol) and dried over P₂O₅ under high vacuum for two days at 40° C. The reaction mixture was flushed with argon and dissolved in dry THF (369.8 mL, Aldrich, sure seal bottle). Diethyl-azodicarboxylate (6.98 mL, 44.36 mmol) was added dropwise to the reaction mixture with the rate of addition maintained such that the resulting deep red coloration is just discharged before adding the next drop. The reaction mixture was stirred for 4 hrs., after which time TLC (EtOAc:hexane, 60:40) indicated that the reaction was complete. The solvent was evaporated in vacuuo and the residue purified by flash column chromatography (eluted with 60:40 EtOAc:hexane), to yield 2′-O-([2-phthalimidoxy)ethyl]-5′-t-butyldiphenylsilyl-5-methyluridine as white foam (21.819 g, 86%) upon rotary evaporation.

### 5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate washed with ice cold CH₂Cl₂, and the combined organic phase was washed with water and brine and dried (anhydrous Na₂SO₄). The solution was filtered and evaporated to afford 2′-O -(aminooxyethyl) thymidine, which was then dissolved in MeOH (67.5 mL). Formaldehyde (20% aqueous solution, w/w, 1.1 eq.) was added and the resulting mixture was stirred for 1 h. The solvent was removed under vacuum and the residue was purified by column chromatography to yield 5′-O-tert-butyldiphenylsilyl-2′-O-[(2-formadoximinooxy) ethyl]-5-methyluridine as white foam (1.95 g, 78%) upon rotary evaporation.

### 5′-O-tert-Butyldiphenylsilyl-2′-O-[N,N dimethylaminooxyethyll-5-methyluridine

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL) and cooled to 10° C. under inert atmosphere. Sodium cyanoborohydride (0.39 g, 6.13 mmol) was added and the reaction mixture was stirred. After 10 minutes the reaction was warmed to room temperature and stirred for 2 h. while the progress of the reaction was monitored by TLC (5% MeOH in CH₂Cl₂). Aqueous NaHCO₃ solution (5%, 10 mL) was added and the product was extracted with EtOAc (2×20 mL). The organic phase was dried over anhydrous Na₂SO₄, filtered, and evaporated to dryness. This entire procedure was repeated with the resulting residue, with the exception that formaldehyde (20% w/w, 30 mL, 3.37 mol) was added upon dissolution of the residue in the PPTS/MeOH solution. After the extraction and evaporation, the residue was purified by flash column chromatography and (eluted with 5% MeOH in CH₂Cl₂) to afford 5′-O-tert-butyldiphenylsilyl-2′-O-[N,N-dimethylaminooxyethyl]-5-methyluridine as a white foam (14.6 g, 80%) upon rotary evaporation.

### 2′-O-(dimethylaminooxyethyl)-5-methyluridine

Triethylamine trihydrofluoride (3.91 mL, 24.0 mmol) was dissolved in dry THF and TEA (1.67 mL, 12 mmol, dry, stored over KOH) and added to 5′-O-tert-butyldiphenylsilyl-2′-O-[N,N-dimethylaminooxyethyl]-5-methyluridine (1.40 g, 2.4 mmol). The reaction was stirred at room temperature for 24 hrs and monitored by TLC (5% MeOH in CH₂Cl₂). The solvent was removed under vacuum and the residue purified by flash column chromatography (eluted with 10% MeOH in CH₂Cl2) to afford 2′-O-(dimethylaminooxyethyl)-5-methyluridine (766 mg, 92.5%) upon rotary evaporation of the solvent.

### 5′-O-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C., co-evaporated with anhydrous pyridine (20 mL), and dissolved in pyridine (11 mL) under argon atmosphere. 4-dimethylaminopyridine (26.5 mg, 2.60 mmol) and 4,4′-dimethoxytrityl chloride (880 mg, 2.60 mmol) were added to the pyridine solution and the reaction mixture was stirred at room temperature until all of the starting material had reacted. Pyridine was removed under vacuum and the residue was purified by column chromatography (eluted with 10% MeOH in CH₂Cl₂ containing a few drops of pyridine) to yield 5′-O-DMT-2′-O-(diimethylamino-oxyethyl)-5-methyluridine (1.13 g, 80%) upon rotary evaporation.

### 5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL), N,N-diisopropylamine tetrazonide (0.29 g, 1.67 mmol) was added and the mixture was dried over P₂O₅ under high vacuum overnight at 40° C. This was dissolved in anhydrous acetonitrile (8.4 mL) and 2-cyanoethyl-N,N,N¹,N¹-tetraisopropylphosphoramidite (2.12 mL, 6.08 mmol) was added. The reaction mixture was stirred at ambient temperature for 4 h under inert atmosphere. The progress of the reaction was monitored by TLC (hexane:EtOAc 1:1). The solvent was evaporated, then the residue was dissolved in EtOAc (70 mL) and washed with 5% aqueous NaHCO₃ (40mL). The EtOAc layer was dried over anhydrous Na₂SO₄, filtered, and concentrated. The residue obtained was purified by column chromatography (EtOAc as eluent) to afford 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%) upon rotary evaporation.

### 2′-(Aminooxyethoxy) nucleoside amidites

2′-(Aminooxyethoxy) nucleoside amidites (also known in the art as 2′-O-(aminooxyethyl) nucleoside amidites) are prepared as described in the following paragraphs. Adenosine, cytidine and thymidine nucleoside amidites are prepared similarly.

### N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutvryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-hydroxyethyl)-5′-O-(4,4′-dimethoxytrityl)guanosine. As before the hydroxyl group may be displaced by N-hydroxyphthalimide via a Mitsunobu reaction, and the protected nucleoside may be phosphitylated as usual to yield 2-N-isobutyryl-6-O-diphenylcarbamoyl-2′-O-([2-phthalmidoxy)ethyl)-5′-O-(4,4′-dimethoxytrityl)guanosine-3′-[(2-cyanoethyl)-N,N-diisopropylphosphoramidite].

### 2′-dimethylaminoethoxyethoxy (2′-DMAEOE) nucleoside amidites

2′-dimethylaminoethoxyethoxy nucleoside amidites (also known in the art as 2′-O-dimethylaminoethoxyethyl, i.e., 2′-O—CH₂—O—CH₂—N(CH₂)₂, or 2′-DMAEOE nucleoside amidites) are prepared as follows. Other nucleoside amidites are prepared similarly.

### 2′-O-(2(2-N,N-dimethylaminoethoxy)ethyl]-5-methyl uridine

2[2-(Dimethylamino)ethoxy]ethanol (Aldrich, 6.66 g, 50 mmol) was slowly added to a solution of borane in tetra-hydrofuran (1 M, 10 mL, 10 mmol) with stirring in a 100 mL bomb. (Caution: Hydrogen gas evolves as the solid dissolves). O²-,2′-anhydro-5-methyluridine (1.2 g, 5 mmol), and sodium bicarbonate (2.5 mg) were added and the bomb was sealed, placed in an oil bath and heated to 155° C. for 26 h then cooled to room temperature. The crude solution was concentrated, the residue was diluted with water (200 mL) and extracted with hexanes (200 mL). The product was extracted from the aqueous layer with EtOAc (3×200 mL) and the combined organic layers were washed once with water, dried over anhydrous sodium sulfate, filtered and concentrated. The residue was purified by silica gel column chromatography (eluted with 5:100:2 MeOH/CH₂Cl₂/TEA) as the eluent. The appropriate fractions were combined and evaporated to afford the product as a white solid.

### 5′-O-dimethoxytrityl-2′-O-[2(2-N,N-dimethylaminoethoxy) ethyl)]-7.5-methyl uridine

To 0.5 g (1.3 mmol) of 2′-O-[2(2-N,N-dimethylamino-ethoxy)ethyl)]-5-methyl uridine in anhydrous pyridine (8 mL), was added TEA (0.36 mL) and dimethoxytrityl chloride (DMT-Cl, 0.87 g, 2 eq.) and the reaction was stirred for 1 h. The reaction mixture was poured into water (200 mL) and extracted with CH₂Cl₂ (2×200 mL). The combined CH₂Cl₂ layers were washed with saturated NaHCO₃ solution, followed by saturated NaCl solution, dried over anhydrous sodium sulfate, filtered and evaporated. The residue was purified by silica gel column chromatography (eluted with 5:100:1 MeOH/CH₂Cl₂/TEA) to afford the product.

### 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) were added to a solution of 5′-O-dimethoxytrityl-2′-O-[2(2-N,N-dimethylaminoethoxy)ethyl))-5-methyluridine (2.17 g, 3 mmol) dissolved in CH₂Cl₂ (20 mL) under an atmosphere of argon. The reaction mixture was stirred overnight and the solvent evaporated. The resulting residue was purified by silica gel column chromatography with EtOAc as the eluent to afford the title compound.

### Example 2

### Oligonucleotide synthesis

Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄oAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No., 5,256,775 or U.S. Pat. No. 5,366,878, herein incorporated by reference.

Alkylphosphonothioate oligonucleotides are prepared as described in International Patent Application Nos. PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

### Oligonucleoside Synthesis

Methylenemethylimino linked oligonucleosides, also identified as MMI linked oligonucleosides, methylenedimethyl-hydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

### PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, and 5,719,262, herein incorporated by reference.

### Example 5

### Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### 2′-O-Me]-[2′-deoxy]-[2′-O-Me] Chimeric Phosphorothioate oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-51-dimethoxytrityl-3′-O-phosphor-amidite for the DNA portion and 5′-dimethoxytrityl-21-O-methyl-3′-O-phosphoramidite for 5′and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

### [2′-O-(2-Methoxyethyl)]-[2′-deoxy]-[2′-O-(Methoxyethyl)] Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiesterl-[2′-deoxyPhosphorothioate]-[2′-O-(2-Methoxyethyl) Phosphodiester] Chimeric Oligonucleotides

[2′-O-(2-methoxyethyl phosphodiesterl-[2′-deoxy phosphorothioate)-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U. S. Pat. No. 5,623,065, herein incorporated by reference.

### Example 6

### Oligonucleotide Isolation

After cleavage from the controlled pore glass solid support and deblocking in concentrated ammonium hydroxide at 55° C. for 12-16 hours, the oligonucleotides or oligonucleosides. are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis were determined by the ratio of correct molecular weight relative to the -16 amu product (±32 ±48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

### Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites. oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

### Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACETM MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

### Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's SA basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblasts (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

b.END Cells:

The mouse brain endothelial cell line b.END was obtained from Dr. Werner Risau at the Max Plank Instititute (Bad Nauheim, Germany). b.END cells were routinely cultured in DMEM, high glucose (Gibco/Life Technologies, Gaithersburg, Md.) supplemented with 10% fetal calf serum (Gibco/Life Technologies, Gaithersburg, Md.). Cells were routinely passaged by trypsinization and dilution when they reached 90% confluence. Cells were seeded into 96-well plates (Falcon-Primaria #3872) at a density of 3000 cells/well for use in RT-PCR analysis.

Treatment with Antisense Compounds:

When cells reached 70% confluency, theft were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 medium containing 3.75 μg/mL LIPOFECTIN™ reagent(Invitrogen Corporation, Carlsbad, Calif.) and the desired concentration of oligonucleotide. After 4-7 hours of treatment, the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 1) which is targeted to human H-ras, or ISIS 18078, (GTGCGCGCGAGCCCGAAATC, SEQ ID NO: 2) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 3, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-Ha-ras (for ISIS 13920) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments.

### Example 10

### Analysis of oligonucleotide inhibition of extracellular-signal-regulated kinase-6 expression

Antisense modulation of extracellular-signal-regulated kinase-6 expression can be assayed in a variety of ways known in the art. For example, extracellular-signal-regulated kinase-6 mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Protein levels of extracellular-signal-regulated kinase-6 can be quantitated in a variety of ways well known in the art, such as immunoprecipitation, Western blot analysis (immunoblotting), ELISA or fluorescence-activated cell sorting (FACS). Antibodies directed to extracellular-signal-regulated kinase-6 can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., (Current Protocols in Molecular Biology, Volume 2, pp. 11.12.1-11.12.9, John Wiley & Sons, Inc., 1997). Preparation of monoclonal antibodies is taught in, for example, Ausubel, F. M. et al., (Current Protocols in Molecular Biology, Volume 2, pp. 11.4.1-11.11.5, John Wiley & Sons, Inc., 1997).

Immunoprecipitation methods are standard in the art and can be found at, for example, Ausubel, F. M. et al., (Current Protocols in Molecular Biology, Volume 2, pp. 10.16.1-10.16.11, John Wiley & Sons, Inc., 1998). Western blot (immunoblot) analysis is standard in the art and can be found at, for example, Ausubel, F. M. et al., (Current Protocols in Molecular Biology, Volume 2, pp. 10.8.1-10.8.21, John Wiley & Sons, Inc., 1997). Enzyme-linked immunosorbent assays (ELISA) are standard in the art and can be found at, for example, Ausubel, F. M. et al., (Current Protocols in Molecular Biology, Volume 2, pp. 11.2.1-11.2.22, John Wiley & Sons, Inc., 1991).

### Example 11

### Poly(A)+ mRNA Isolation

Poly(A)+ mRNA was isolated according to Miura et al., (Clin. Chem., 1996, 42, 1758-1764). Other methods for poly(A)+ mRNA isolation are taught in, for example, Ausubel, F. M. et al., (Current Protocols in Molecular Biology, Volume 1, pp. 4.5.1-4.5.3, John Wiley & Sons, Inc., 1993). Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes i at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Example 12

### Total RNA Isolation

Total RNA was isolated using an RNEASY ₉₆™ kit and buffers purchased from Qiagen Inc. (Valencia, Calif.) following the manufacturer's recommended procedures. Briefly, for cells grown on 96-well plates, growth medium was removed from the cells and_each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 170 μL water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

### Real-time Quantitative PCR Analysis of extracellular-signal-regulated kinase-6 mRNA Levels

Quantitation of extracellular-signal-regulated kinase-6 mPNA levels was determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA;, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, CA). RT-PCR reactions were carried out by adding 20 ×L PCR cocktail (2.5× PCR buffer (—MgCl₂), 6.6 mM MgCl₂, 375 μM each of DATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution. The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ reagent (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent from Molecular Probes. Methods of RNA quantification by RiboGreen™ reagent are taught in Jones, L. J. et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor™ 4000 instrument (PE Applied Biosystems) with excitation at 480nm and emission at 520nm.

Probes:and primers to human extracellular-signal-regulated kinase-6 were designed to hybridize to a human extracellular-signal-regulated kinase-6 sequence, using published sequence information (GenBank accession number X79483.1, incorporated herein as SEQ ID NO:4). For human extracellular-signal-regulated kinase-6 the PCR primers were: forward primer: CTCGTTGCCACCTTGACCTT (SEQ ID NO: 5) reverse primer: TGGAACCCGGGCGTCT (SEQ ID NO: 6) and the PCR probe was: FAM-TTGCATCCCAAGGCATCCATCAGA-TAMRA (SEQ ID NO: 7) where FAM is the fluorescent dye and TAMRA is the quencher dye. For human GAPDH the PCR primers were: forward primer: GAAGGTGAAGGTCGGAGTC(SEQ ID NO:8) reverse primer: GAAGATGGTGATGGGATTTC (SEQ ID NO:9) and the PCR probe was: 5′ JOE-CAAGCTTCCCGTTCTCAGCC-TAMRA 3′ (SEQ ID NO: 10) where JOE is the fluorescent reporter dye and TAMRA is the quencher dye.

### Example 14

### Northern blot analysis of extracellular-signal-regulated kinase-6 mRNA levels

Eighteen hours after antisense treatment, cell monolayers were washed twice with cold PBS and lysed in 1 mL RNAZOL™ reagent(TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 instrument (Stratagene, Inc, La Jolla, Calif.) and then probed using QUICKHYB™ hybridization solution (Stratagene,: La Jolla, Calif.) using manufacturer's recommendations for stringent conditions.

To detect human extracellular-signal-regulated kinase-6, a human extracellular-signal-regulated kinase-6 specific probe was prepared by PCR using the forward primer CTCGTTGCCACCTTGACCTT (SEQ ID NO: 5) and the reverse primer TGGAACCCGGGCGTCT (SEQ ID NO: 6). To normalize for variations in loading and transfer efficiency membranes were stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ instrument and IMAGEQUANT™ Software V3.3 (Molecular Dynamics, Sunnyvale, Calif.). Data was normalized to GAPDH levels in untreated controls.

### Example 15

### Antisense inhibition of human extracellular-signal-regulated kinase-6 expression by chimeric phosphorothioate oligonucleotides having 2′-MOE wings and a deoxy gap

In accordance with the present invention, a series of oligonucleotides were designed to target different regions of the human extracellular-signal-regulated kinase-6 RNA, using published sequences (GenBank accession number X79483.1, incorporated herein as SEQ ID NO: 4). The oligonucleotides are shown in Table 1. “Target site” indicates the first (5′-most) nucleotide number on the particular target sequence to which the oligonucleotide binds. All compounds in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines.

The compounds were analyzed for their effect on human extracellular-signal-regulated kinase-6 mRNA levels by quantitative real-time PCR as described in other examples herein. Data are averages from two experiments in which T-24 cells were treated with the antisense oligonucleotides of the present invention. The positive control for each datapoint is identified in the table by sequence ID number. The positive control for each datapoint is identified in the table by sequence ID number. If present, “N.D.” indicates “no data”.

TABLE 1

Inhibition of human extracellular-signal-regulated kinase-6
mRNA levels by chimeric phosphorothioate oligonucleotides
having 2′-MOE wings and a deoxy gap

| | | | | | | | |
|---|---|---|---|---|---|---|---|
| | | TARGET | | | | | CONTROL |
| | | SEQ ID | TARGET | | % | SEQ | SEQ ID |
| ISIS # | REGION | NO | SITE | SEQUENCE | INHIB | ID NO | NO |

| | | | | | | | |
|---|---|---|---|---|---|---|---|
| 157011 | Coding | 4 | 432 | ccttcatcatctggtacacg | 6 | 11 | 2 |
| 157012 | Coding | 4 | 724 | tccttcagctggtccaggtg | 55 | 12 | 2 |
| 157013 | 3′UTR | 4 | 1412 | ccaccagctctgaggtttct | 63 | 13 | 2 |
| 157014 | Start | 4 | 25 | ggagagctcatggcaggccc | 8 | 14 | 2 |
| | Codon | | | | | | |
| 157015 | Coding | 4 | 263 | gtggcgcatgtgcttgagca | 48 | 15 | 2 |
| 157016 | Coding | 4 | 433 | cccttcatcatctggtacac | 9 | 16 | 2 |
| 157017 | Coding | 4 | 314 | atccagggtctcatcaggag | 48 | 17 | 2 |
| 157018 | Stop | 4 | 1136 | cccggagcccagagatcttc | 78 | 18 | 2 |
| | Codon | | | | | | |
| 157019 | 3′UTR | 4 | 1474 | ctgtccttcctctgcatggc | 0 | 19 | 2 |
| 157020 | Coding | 4 | 410 | gaactggatccggtcctcgc | 55 | 20 | 2 |
| 157021 | Coding | 4 | 534 | ggccgaagtccaggatcttc | 61 | 21 | 2 |
| 157022 | 3′UTR | 4 | 1499 | tgagttggtgccctgctccc | 45 | 22 | 2 |
| 157023 | 3′UTR | 4 | 1232 | tctgatggatgccttgggat | 85 | 23 | 2 |
| 157024 | Coding | 4 | 427 | atcatctggtacacgaggaa | 56 | 24 | 2 |
| 157025 | Coding | 4 | 394 | tcgcctagcttctcatgttt | 46 | 25 | 2 |
| 157026 | Coding | 4 | 837 | aggcaaaatccttcttctcc | 39 | 26 | 2 |
| 157027 | 3′UTR | 4 | 1235 | tgctctgatggatgccttgg | 38 | 27 | 2 |
| 157028 | 3′UTR | 4 | 1612 | gctacaaaagggtctatttc | 52 | 28 | 2 |
| 157029 | Coding | 4 | 545 | ctgcctggccaggccgaagt | 33 | 29 | 2 |
| 157030 | Coding | 4 | 933 | aaagaaactcgcctgcagtc | 0 | 30 | 2 |
| 157031 | 3′UTR | 4 | 1435 | gaggccaaggctgatctgga | 23 | 31 | 2 |
| 157032 | Coding | 4 | 419 | gtacacgaggaactggatcc | 56 | 32 | 2 |
| 157033 | Coding | 4 | 338 | caccaggtaaaagtccgtga | 25 | 33 | 2 |
| 157034 | Coding | 4 | 1015 | tcaaagtagtcaaaggagtc | 0 | 34 | 2 |
| 157035 | Coding | 4 | 197 | gggccgatacagcttcttga | 46 | 35 | 2 |
| 157036 | 3′UTR | 4 | 1420 | ctggagccccaccagctctg | 61 | 36 | 2 |
| 157037 | Coding | 4 | 769 | cgctgcacaaactcagccgg | 59 | 37 | 2 |
| 157038 | Coding | 4 | 807 | ggcccttcatgtagttcttg | 36 | 38 | 2 |
| 157039 | Coding | 4 | 1002 | aggagtcatcatacttctgg | 54 | 39 | 2 |
| 157040 | 3′UTR | 4 | 1421 | tctggagccccaccagctct | 30 | 40 | 2 |
| 157041 | 3′UTR | 4 | 1509 | gggatgtccctgagttggtg | 52 | 41 | 2 |
| 157042 | 3′UTR | 4 | 1544 | ggtgcaggaaggtccactga | 64 | 42 | 2 |
| 157043 | Coding | 4 | 380 | atgtttcatgagcttgccca | 54 | 43 | 2 |
| 157044 | Coding | 4 | 443 | atacctcagccccttcatca | 26 | 44 | 2 |
| 157045 | Coding | 4 | 369 | gcttgcccaggtcggtgccc | 62 | 45 | 2 |
| 157046 | 3′UTR | 4 | 1198 | aggtcaaggtggcaacgaga | 75 | 46 | 2 |
| 157047 | Coding | 4 | 798 | tgtagttcttggcctcatcg | 56 | 47 | 2 |

As shown in Table 1, SEQ ID NOs 12, 13, 15, 17, 18, 20, 23, 24, 25, 28, 32, 35, 36, 37, 39, 41, 42, 43, 45, 46 and 47 demonstrated at least 40% inhibition of human extracellular-signal-regulated kinase-6 expression in this assay. The target sites to which these antisense sequences are complementary are herein referred to as “illustrative target regions” and are illustrative sites for targeting by compounds of the present invention. These same target regions are shown in Table 2. The sequences represent the reverse complement of the antisense compounds shown in Table 1. “Target site” indicates the first (5′-most) nucleotide number of the corresponding target nucleic acid. Also shown in Table 2 is the species in which each of these target regions was found.

TABLE 2

Sequence and position of illustrative target regions
identified in extracellular-signal-regulated kinase-6.

| | | | | | | |
|---|---|---|---|---|---|---|
| | TARGET | | | REV | | |
| SITE | SEQ ID | TARGET | | COMP OF | | SEQ ID |
| ID | NO | SITE | SEQUENCE | SEQ ID | ACTIVE IN | NO |

| | | | | | | |
|---|---|---|---|---|---|---|
| 72512 | 4 | 724 | cacctggaccagctgaagga | 12 | H. sapiens | 48 |
| 72513 | 4 | 1412 | agaaacctcagagctggtgg | 13 | H. sapiens | 49 |
| 72515 | 4 | 263 | tgctcaagcacatgcgccac | 15 | H. sapiens | 50 |
| 72517 | 4 | 314 | ctcctgatgagaccctggat | 17 | H. sapiens | 51 |
| 72518 | 4 | 1136 | gaagatctctgggctccggg | 18 | H. sapiens | 52 |
| 72520 | 4 | 410 | gcgaggaccggatccagttc | 20 | H. sapiens | 53 |
| 72521 | 4 | 534 | gaagatcctggacttcggcc | 21 | H. sapiens | 54 |
| 72522 | 4 | 1499 | gggagcagggcaccaactca | 22 | H. sapiens | 55 |
| 72523 | 4 | 1232 | atcccaaggcatccatcaga | 23 | H. sapiens | 56 |
| 72524 | 4 | 427 | ttcctcgtgtaccagatgat | 24 | H. sapiens | 57 |
| 72525 | 4 | 394 | aaacatgagaagctaggcga | 25 | H. sapiens | 58 |
| 72528 | 4 | 1612 | gaaatagacccttttgtagc | 28 | H. sapiens | 59 |
| 72532 | 4 | 419 | ggatccagttcctcgtgtac | 32 | H. sapiens | 60 |
| 72535 | 4 | 197 | tcaagaaqctqtatcggccc | 35 | H. sapiens | 61 |
| 72536 | 4 | 1420 | cagagctggtggggctccag | 36 | H. sapiens | 62 |
| 72537 | 4 | 769 | ccggctgagtttgtgeagcg | 37 | H. sapiens | 63 |
| 72539 | 4 | 1002 | ccagaagtatgatgactcct | 39 | H. sapiens | 64 |
| 72541 | 4 | 1509 | caccaactcagggacatccc | 41 | H. sapiens | 65 |
| 72542 | 4 | 1544 | tcagtggaccttcctgcacc | 42 | H. sapiens | 66 |
| 72543 | 4 | 380 | tgggcaagctcatgaaacat | 43 | H. sapiens | 67 |
| 72545 | 4 | 369 | gggcaccgacctgggcaagc | 45 | H. sapiens | 68 |
| 72546 | 4 | 1198 | tctcgttgccatcttgacct | 46 | H. sapiens | 69 |
| 72547 | 4 | 798 | cgatgaggccaagaactaca | 47 | H. sapiens | 70 |

As these “illustrative target regions” have been found by experimentation to be open to, and accessible for, hybridization with the antisense compounds of the present invention, one of skill in the art will recognize or be able to ascertain, using no more than routine experimentation and provided with the teachings of this invention, further embodiments of the invention that encompass other compounds that specifically hybridize to these sites and consequently inhibit the expression of extracellular-signal-regulated kinase-6.

### Example 16

### Western blot analysis of extracellular-signal-regulated kinase-6 protein levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to extracellular-signal-regulated kinase-6 is used, with a radiolabeled or fluorescently labeled secondary antibody directed against the primary antibody species. Bands are visualized using a PHOSPHORIMAGE™ instrument (Molecular Dynamics, Sunnyvale Calif.).

### Example 17

### Treatment of Angiogenic Disease: Breast Cancer

Breast carcinoma is the most common type of tumors in women over 40 years age and a leading cause of deaths. The beneficial effect on patients with breast cancer with the extracellular-signal-regulated kinase-6 inhibitor may be shown in the following clinical trials:

In a first clinical trial, 5 patients suffering from metastatic breast carcinoma are studied, who have no previous systemic treatment of metastasis (adjuvant treatment is ignored) and have easy access to their veins. The patients have PS 0 or 1 and may be post-menopausal.

The extracellular-signal-regulated kinase-6 inhibitor may be continuously administered parenterally, e.g. s.c. by means of a pump at the rate of e.g. 0.5 to 2 mg per 24 hours, over at least 3 days. The growth factor IGF profile is determined and the levels are found to be reduced.

A second clinical trial may be effected as follows:

In a second trial the extracellular-signal-regulated kinase-6 inhibitors are administered to at least 14 patients having breast cancer and the extent and duration of the response are determined.

Patients are included who have breast cancer as evidenced by histological biopsy (glandular analysis-EOA) They present a metastatic illness and/or loco-regional localization that is measurable and evaluable. If desired, patients resistant to other treatment to conventional therapy such as surgery, radiotherapy, other chemotherapy and/or hormone therapy are included.

The patients present at least one target (identifier), on X-ray analysis, which is measurable or evaluable such as a primitive metastatic tumor that is cutaneous or sub-cutaneous. It may be gangliar or visceral. Preferably the patients have lesions that have progressed within the month preceding the trial and have an estimated survival time of at least 3 months.

Preferably the trial excludes: patients in which the sole criteria for diagnosing breast cancer are biological modifications; patients administered with an embroynic carcinoma antigen pathology; patients with ascitis, a pleural |effusion, a pulmonary carcinoma lymphangitis, or an osseous localization as sole metastatic manifestation; patients treated on a unique tumoral target by radiotherapy less than eight weeks before inclusion in the study (they are eligible however if evidence of progression during this time); patients with a unique cerebral localization; patients presenting another malignant tumor with the exception of:a carcinoma in situ in the cervix uteri or a spino- or basocellular skin cancer; and patients not able to attend regular consultations.

With these exclusions the efficacy of the extracellular-signal-regulated kinase-6 inhibitors may be followed more clearly. The extracellular-signal-regulated kinase-6 inhibitors may be used in the methods of treatment of the invention.

The extracellular-signal-regulated kinase-6 inhibitors may be administered at the same dosage as, or at a lower dosage than, in the first trial. Preferably it is administered in two doses, one in the morning and one in the evening. The treatment duration is at least 3 months or until complete remission. The response may be followed by conventional methodology, e.g. according to IUCC response criteria, e.g. progression, stabilization, partial or complete remission. The evaluation is effected e.g. on day 0, 15, 45, 60 and 90.

A third clinical trial may be effected as follows:

Patients with advanced breast cancer are included. The patients have progressive disease and measurable and/or evaluable parameters according to criteria of the IUCC (i.e. appearance of new lesions or growth of existing metastatic lesions) not responding to primary hormonal and/or cytotoxic therapy. As in the above indicated second clinical trial, the third trial preferably also excludes patients with previous or concurrent malignancies at other sites, with the exception of cone biopsied in situ carcinoma of the cervix uteri and adequately treated basal or squamous cell carcinoma of the skin.

The extracellular-signal-regulated kinase-6 inhibitor may be administered at the same dosage as or at a lower dosage than in the second trial. Preferably the inhibitors are administered parenterally, e.g. subcutaneous, particularly in a continuous subcutaneous way by means of a portable syringe pump (infusion pump). Treatment is for at least 2 months or until complete remission. The response may be followed by conventional methodology, e.g. according to IUCC response criteria. The evaluation is effected, e.g. on day 0, 30 and 60. All lesions are measured at each assessment. Alternatively, when multiple lesions are present, a representative number of 5 lesions may be selected for measurement. Regression of the lesions is the sum of the products of the diameters of each individual lesion or those selected for study, which decreases by 50% or more.

### Example 18

### Treatment of Angiogenic Disease: Melanoma

In an in vivo test, Meth-A sarcoma and melanoma cells (1×10⁶) are inoculated subcutaneously in 0.1 ml saline in the same position of the dorsal skin of C3H mice (n=20). On the same day, the mice receive orally either extracellular-signal-regulated kinase-6 inhibitor, at 100 mg per kg in body weight, suspended in 300 uL of olive oil (n=10) or 300 uL olive oil alone (n=10). This treatment is carried out every day and the diameters of the tumors are monitored every second day. On day 12 the mice are sacrificed and the tumor weights are measured.

Meth-A sarcoma tumor growth in mice treated with extracellular-signal-regulated kinase-6 inhibitor is slower than in control mice. The weight (grams) of both the Meth-A sarcoma and melanoma tumors on day 12 is measured, and the mice treated with extracellular-signal-regulated kinase-6 inhibitor have lower tumor mass. In a small number of control and 2-methoxyestradiol mice, the dorsal skin, together with the tumor, are excised and the angiogenesis within the subcutaneous fascia in the control and treated mice is visualized with indian ink. Apart from their marginally lower weight, the treated mice exhibit no apparent signs of toxicity and all are alive after 12 days of daily treatment.

Extracellular-signal-regulated kinase-6 inhibitor thus has potent pharmacological properties that may be applied in the treatment angiogenic diseases, including solid tumors.

### Example 19

### Methods of Inhibiting Angiogenesis

Angiogenesis is the growth of new blood vessels (veins & arteries) by endothelial cells. This process is important in the development of a number of human diseases, and is believed to be particularly important in regulating the growth of solid tumors. Without new vessel formation it is believed that tumors will not grow beyond a few millimeters in size. In addition to their use as anticancer agents, inhibitors of angiogenesis have potential for the treatment of diabetic retinopathy, cardiovascular disease, rheumatoid arthritis and psoriasis.

During the process of angiogenesis, endothelial cells perform several distinct functions, including the degradation of the extracellular matrix (ECM), migration, proliferation and the formation of tube-like structures. Various genes may regulate some of these processes in primary human umbilical vein endothelial cells (HUVECs). The extracellular-signal-regulated kinase-6 inhibitor employed in this assay is an antisense oligomer ISIS No. 157018 (SEQ ID NO: 18); and the control (or negative control) employed in this assay is the mixed sequence 20-mer negative oligonucleotide control, NNNNNNNNNNNNNNNNNNNN, incorporated herein as SEQ ID NO: 71.

(A) Matrix MetalloProteinase (MMP) Activity Assay

During angiogenesis, endothelial cells need to be able to degrade the extracellular matrix so they can migrate and form new vessels. Endothelial cells secrete matrix metalloproteinases (MMPs) in order to accomplish this degradation. MMPs are a family of zinc-dependent endopeptidases that fall into eight distinct classes: five are secreted and three are membrane-type MMPs (MT-MMPs). MMPs exert these effects by cleaving a diverse group of substrates, which include not only structural components of the extracellular matrix, but also growth-factor-binding proteins, growth-factor precursors, receptor tyrosine kinases, cell-adhesion molecules and other proteinases. In this assay the activity of MMPs secreted into the media above antisense oligonucleotide-treated HUVECs is measured.

MMP activity in the media above HUVECs is measured using the EnzChek™ Gelatinase/Collagenase Assay Kit (Molecular Probes, Eugene, Oreg.). HUVECs are plated at 3000 cells/well in 96-well plates. One day later, cells are transfected with antisense oligonucleotides according to standard published procedures (Monia et al., J Biol Chem. 1993 268(19) :14514-22) with 75 nM oligonucleotide in lipofectin (Gibco, Grand Island, N.Y.). Antisense oligonucleotides are tested in triplicate on each 96-well plate, except for positive and negative antisense controls, which are measured up to six times per plate. Twenty hours post-transfection, MMP production is stimulated by the addition of recombinant human vascular endothelial growth factor (VEGF). Fifty hours post-transfection, a p-aminophenylmercuric acetate (APMA; Sigma-Aldrich, St. Louis, Mo.) solution is added to each well of a Corning-Costar 96-well clear bottom plate (VWR International, Brisbane, Calif.). The APMA solution is used to promote cleavage of inactive MMP precursor proteins (Nagase et al., Biomed Biochim Acta, 1991 50(4-6) :749-54).

Media above the HUVECs is then transferred to the wells. After 30 minutes, the quenched, fluorogenic MMP cleavage substrate is added, and baseline fluorescence is read immediately at 485 nm exitation/530 nm emission. Following an overnight incubation at 37° C. in the dark, plates are read L again to determine the amount of fluorescence, which corresponds to MMP activity. Total protein from HUVEC lysates is used to normalize the readings, and MMP activities±standard deviation are expressed relative to transfectant-only controls.

(B) Endothelial Tube Formation Assay

Angiogenesis is stimulated by numerous factors that promote interaction of endothelial cells with each other and with extracellular matrix molecules, resulting in the formation of capillary tubes. This morphogenic process is necessary for the delivery of oxygen to nearby tissues and plays an essential role in embryonic development, wound healing, and tumor growth. Moreover, this process can be reproduced in tissue culture by the formation of tube-like structures by endothelial cells. There are several different variations of the assay that use different matrices, such as collagen I (Kanayasu, 1991], Matrigel [Yamagishi, 1997] and fibrin [Bach, 1998] as growth substrates for the cells. In this assay, UVECs are plated on a matrix derived from the Engelbreth-Holm-Swarm mouse tumor, which is very similar to Matrigel [Kleinman, 1986; Madri, 1986]. Untreated HUVECs form tube-like structures when grown on this substrate. Loss of tube formation in vitro has been correlated with the inhibition of angiogenesis in vivo (Carmeliet et al., Nature 2000 407:249-257; and Zhang et al., Cancer Research 2002 62-:2034-42), which supports the use of in vitro tube formation as an endpoint for angiogenesis.

The Tube Formation Assay is performed using an In vitro Angiogenesis Assay Kit (Chemicon International, Temecula, Calif.), or growth factor reduced Mortigel (BD Biosciences, Bedford, Mass.). Cells are plated and transfected with extracellular-signal-regulated kinase-6 inhibitors (antisense oligonucleotides) as described for the MMP activity assay, except cells are plated at 4000 cells/well. Fifty hours post-transfection, cells are transferred to 96-well plates coated with ECMatrix reagent (Chemicon International) or growth factor depleted matrigel. Under these conditions, untreated HUVECs form tube-like structures. After an overnight incubation at 37° C., treated and untreated cells are inspected by light microscopy. Individual wells are assigned discrete scores from 1 to 5 depending on the extent of tube formation. A score of 1 refers to a well with no tube formation while a score of 5 is given to wells where all cells are forming an extensive tubular network.

As calculated from the assigned discreet scores, cells treated with extracellular-signal-regulated kinase-6 inhibitors had tube formation score reduction of about 50% as compared to lipid-treated cells. Thus, it is shown that extracellular-signal-regulated kinase-6 inhibitors can inhibit angiogenesis.

(C) RNA Expression Levels of Angiogenic Genes

Endothelial cells must regulate the expression of many genes in order to perform the functions necessary for angiogenesis. This gene regulation has been the subject of intense scrutiny, and many genes have been identified as being important for the angiogenic phenotype. The expression levels of four genes, previously identified as being highly expressed in angiogenic endothelial cells, are measured (i.e., Integrin beta 3, endoglin/CD105, TEMS and MMP-14/MT-MMP1).

Integrin beta 3 is part of a family of heterodimeric transmembrane receptors that consist of alpha and beta subunits. Each subunit recognizes a unique set of ECM ligands, thereby allowing cells to transmit angiogenic signals from the extracellular matrix. Integrin beta 3 is prominently expressed on proliferating vascular endothelial cells, and it plays roles in allowing new blood vessels to form at tumor sites as well as allowing the epithelial cells of breast tumors to spread. Blockage of Integrin alpha 3 with monoclonal antibodies or low molecular weight antagonists inhibits blood vessel formation in a variety of in vivo models, including tumor angiogenesis and neovascularization during oxygen-induced retinopathy.

Endoglin is a Transforming Growth Factor receptor-associated protein highly expressed on endothelial cells, and resent on some leukemia cells and minor subsets of bone arrow cells. Its expression is upregulated in endothelial cells of angiogenic tissues and is therefore used as a prognostic indicator in various tumors. Endoglin functions as an ancillary receptor influencing binding of the Transforming Growth Factor beta (TGF-beta) family of ligands to signaling receptors, thus mediating cell survival. Mutations of the endoglin gene result in a genetic disease of the vasculature, i.e., Hereditary Haemorrhagic Telangiectasia (HHT), which is characterized by bleeding from malformed blood vessels. Defective signaling by different TGF-beta ligands and receptors is thought to be involved.

Tumor endothelial marker 5 (TEMS) is a putative 7-pass transmembrane protein (GPCR) for which EST sequence but no other information is available. The mRNA transcript, designated KIAA1531, encodes one of many tumor endothelium markers (TEMs) that display elevated expression (greater than 10-fold) during tumor angiogenesis. TEM5 is coordinately expressed with other TEMs on tumor endothelium in humans and mice.

MMP-14, a membrane-type MMP (MT-MMP) covalently linked to the cell membrane, is involved in matrix detachment and migration. MMP-14 is thought to promote tumor angiogenesis; antibodies directed against the catalytic domain of MMP-14 block endothelial-cell migration, invasion and capillary tube formation in vitro. MMP-14 can degrade the fibrin matrix that surrounds newly formed vessels, allowing the endothelial cells to invade further into the tumor tissue. MMP-14 null mice have impaired angiogenesis during development, further demonstrating the role of MMP-14 in angiogenesis.

Cells are plated and transfected as described for the MMP activity assay. Twenty hours post-transfection, cells are stimulated with recombinant human VEGF. Total RNA is harvested 52 hours post-transfection, and the amount of total RNA from each sample is determined using a Ribogreen Assay (Molecular Probes, Eugene, Oreg.). Real-time PCR is performed on the total. RNA using primer/probe sets for four Angiogenic Hallmark Genes: integrin beta 3, endoglin, Tumor endothelial marker 5 (TEM5) and Matrix Metalloproteinase 14 (MMPl4/MT1-MMP). Expression levels for each gene are normalized to total RNA, and values are expressed relative to controls.

Cells treated with extracellular-signal-regulated kinase-6 inhibitors had integrin beta 3 mRNA reduced by 50% and extracellular-signal-regulated kinase-6 mRNA levels were reduced by 75%.

### Example 20

### Effect of antisense inhibition of human Extracellular-signal-regulated kinase-6 expression on migration of HUVEC cells through the fibronectin chamber

An important function of endothelial cells during the process of angiogenesis is the ability to migrate through the extracellular matrix. This process is mimicked in vitro by using an assay in which the ability of cells to get from one side of a Boyden chamber to the other is measured. The ability of HUVEC cells to migrate through the fibronectin chamber was investigated as a function of inhibition of extracellular-signal-regulated kinase-6 expression with ISIS 157018 (SEQ ID NO: 18) and ISIS 157046 (SEQ ID NO: 46).

HUVEC cells were transfected with oligonucleotides for 4 hours then the oligonucleotide-containing media was replaced with regular growth media and the cells were allowed to grow for 44 hours. Forty-eight hours after the start of transfection, the cells were harvested with EDTA/trypsin and resuspended in media containing 5% BSA at 250,000 cells/ml. Meanwhile, each well of a 96-well migration plate (Chemicon International, Temecula, Calif., Catalog #ECM510) was coated on the bottom by adding 150 μl of media with 0.5% bovine serum albumin (BSA)±10 μg/ml of extracellular matrix protein (e.g. fibronectin) and the plate was incubated for 2 hours at room temperature. The protein-containing media was removed from below the wells of the 96-well migration plate and replaced with media containing 5% BSA±growth factors (e.g. complete growth media). To the top of the well was added 100 μl of the HUVEC cell suspension. The plate was incubated between 4 to 16 hours at 37° C., after which the cells and-media were removed from inside the wells by tamping out the media. The plate was then placed into a tray with 150 μl of cell detachment solution (Chemicon International, Temecula, Calif.) for 30 minutes at 37° C. followed by 50 μl of cell lysis buffer/Cyquant (Chemicon International) for 15 minutes at room temperature. Finally, the plate was read on a fluorometric plate reader with a 480/520 nm filter set.

To calculate the results, the values from wells coated only with BSA, and containing only BSA in the media, are set as background and are subtracted from all other values. All values are expressed relative to values determined for the control group (treated with a the mixed 20-mer negative oligonucleotide control, ISIS 29848, NNNNNNNNNNNNNNNNNNNN, wherein N is A, T, C, or G, incorporated herein as SEQ ID NO: 71).

Treatment with ISIS 157018 or ISIS 157046 inhibited the migration of HUVEC cells through the fibronectin chamber by 70% and 55%, respectively. This indicates that inhibition of extracellular-signal-regulated kinase-6 expression in endothelial cells could be an effective means of blocking angiogenesis in vivo.

### Example 21

### Antisense inhibition of mouse extracellular-signal-regulated kinase-6 expression by chimeric phosphorothioate oligonucleotides having 2′-MOE wings and a deoxy gap

In accordance with the present invention, a second series of antisense compounds were designed to target different regions of the mouse extracellular-signal-regulated kinase-6 RNA, using published sequences (GenBank accession number AK011286.1, incorporated herein as SEQ ID NO: 72, GenBank accession number NM_{—}013871.1, incorporated herein as SEQ ID NO: 73, GenBank accession number BI737344.1, incorporated herein as SEQ ID NO: 74, GenBank accession number BU151879.1, incorporated herein as SEQ ID NO: 75, GenBank accession number BC002134.1, incorporated herein as SEQ ID NO: 76, and GenBank accession number BE303845.1, incorporated herein as SEQ ID NO: 77). The compounds are shown in Table 3. “Target site” indicates the first (5′-most) nucleotide number on the particular target nucleic acid to which the compound binds. All compounds in Table 3 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on mouse extracellular-signal-regulated kinase-6 mRNA levels by quantitative real-time PCR as described in other examples herein. Data are averages from three experiments in which b.END cells were treated with the antisense oligonucleotides of the present invention. The positive control for each datapoint is identified in the table by. sequence ID number. If present, “N.D.” indicates “no data”.

Probes and primers to mouse extracellular-signal-regulated kinase-6 were designed to hybridize to a mouse extracellular-signal-regulated kinase-6 sequence, using published sequence information (GenBank accession number AK011286.1, incorporated herein as SEQ ID NO:72). For mouse extracellular-signal-regulated kinase-6 the PCR primers were: forward primer: GGCAAATTCAACGGCACAGT (SEQ ID NO:78) reverse primer: CCAGGTGGAAGACTTGGGATAC (SEQ ID NO: 79) and the PCR probe was: FAM-CACCCCGACAGTGCCTGGAGC-TAMRA (SEQ ID NO: 80) where FAM is the fluorescent reporter dye and TAMRA is the quencher dye. For mouse GAPDH the PCR primers were: forward primer: GGCAAATTCAACGGCACAGT(SEQ ID NO:81) reverse primer: GGGTCTCGCTCCTGGAAGAT(SEQ ID NO:82) and the PCR probe was: 5′ JOE-AAGGCCGAGAATGGGAAGCTTGTCATC-TAMRA 31 (SEQ ID NO: 83), where JOE is the fluorescent reporter dye and TAMRA is the quencher dye.

To detect mouse extracellular-signal-regulated kinase-6, mouse extracellular-signal-regulated kinase-6 specific probe was prepared by PCR using the forward primer TCTCTGGAAAGGAATCCTGGTTA (SEQ ID NO: 78) and the reverse primer CCAGGTGGAAGACTTGGGATAC (SEQ ID NO: 79). To normalize for variations in loading and transfer efficiency membranes were stripped and probed for mouse glyceraldehyde-3-phosphate dehydrogenase (GAPDH) RNA (Clontech, Palo Alto, Calif.).

TABLE 3

Inhibition of mouse extracellular-signal-regulated kinase-6
mRNA levels by chimeric phosphorothioate oligonucleotides
having 2′-MOE wings and a deoxy gap

| | | | | | | | |
|---|---|---|---|---|---|---|---|
| | | TARGET | | | | | CONTROL |
| | | SEQ ID | TARGET | | % | SEQ ID | SEQ ID |
| ISIS # | REGION | NO | SITE | SEQUENCE | INHIB | NO | NO |

| | | | | | | | |
|---|---|---|---|---|---|---|---|
| 216404 | Coding | 72 | 799 | tcatctcactgtctgcctgc | 85 | 84 | 1 |
| 312000 | 5′UTR | 73 | 87 | ccgggtcactgcgtggcagg | 80 | 85 | 1 |
| 312001 | Start | 73 | 158 | gggagctcatccccagccgg | 85 | 86 | 1 |
| | Codon | | | | | | |
| 312002 | Coding | 73 | 184 | gtaaaagcccttgcgggcgg | 55 | 87 | 1 |
| 312003 | Coding | 73 | 189 | tggcggtaaaagcccttgcg | 78 | 88 | 1 |
| 312004 | Coding | 73 | 195 | acctcctggcggtaaaagcc | 74 | 89 | 1 |
| 312005 | Coding | 73 | 200 | tggtcacctcctggcggtaa | 79 | 90 | 1 |
| 312006 | Coding | 73 | 209 | aggccgttttggtcacctcc | 92 | 91 | 1 |
| 312007 | Coding | 73 | 219 | cgcacctcccaggccgtttt | 84 | 92 | 1 |
| 312008 | Coding | 73 | 252 | gagccaacgggctgcaggtc | 77 | 93 | 1 |
| 312009 | Coding | 73 | 257 | caccagagccaacgggctgc | 84 | 94 | 1 |
| 312010 | Coding | 73 | 267 | gcaccataggcaccagagcc | 90 | 95 | 1 |
| 312011 | Coding | 73 | 272 | acactgcaccataggcacca | 83 | 96 | 1 |
| 312012 | Coding | 73 | 280 | tgcagagcacactgcaccat | 77 | 97 | 1 |
| 312013 | Coding | 73 | 290 | ggctgtctactgcagagcac | 69 | 98 | 1 |
| 312014 | Coding | 73 | 295 | agtgcggctgtctactgcag | 73 | 99 | 1 |
| 312015 | Coding | 73 | 300 | ttgccagtccggctgtctac | 75 | 100 | 1 |
| 312016 | Coding | 73 | 305 | ccttgttgccagtgcggctg | 78 | 101 | 1 |
| 312017 | Coding | 73 | 310 | ggccaccttgttgccagtgc | 86 | 102 | 1 |
| 312018 | Coding | 73 | 371 | actctctgtaggcgcgcttg | 87 | 103 | 1 |
| 312019 | Coding | 73 | 376 | gcgcaactctctgtaggcgc | 87 | 104 | 1 |
| 312020 | Coding | 73 | 382 | gaggaggcgcaactctctgt | 84 | 105 | 1 |
| 312021 | Coding | 73 | 389 | tgtgtttgaggaggcgcaac | 73 | 106 | 1 |
| 312022 | Coding | 73 | 399 | tcgtggcgcatgtgtttgag | 81 | 107 | 1 |
| 312023 | Coding | 73 | 468 | aggtagaagtctgtgaagtc | 65 | 108 | 1 |
| 312024 | Coding | 73 | 473 | tcaccagatagaagtctgtg | 61 | 109 | 1 |
| 312025 | Coding | 73 | 487 | gcccatgaatggcatcacca | 86 | 110 | 1 |
| 312026 | Coding | 73 | 492 | tcagtgcccatgaatggcat | 81 | 111 | 1 |
| 312027 | Coding | 73 | 540 | tggattctgtcttcactcag | 81 | 112 | 1 |
| 312028 | Coding | 73 | 545 | gaaactggattctgtcttca | 79 | 113 | 1 |
| 312029 | Coding | 73 | 550 | cacaagaaactggattctgt | 84 | 114 | 1 |
| 312030 | Coding | 73 | 555 | tgatacacaagaaactggat | 84 | 115 | 1 |
| 312031 | Coding | 73 | 681 | gcctgcctggcaaggccaaa | 83 | 116 | 1 |
| 312032 | Coding | 73 | 690 | tcactgtctgcctgcctggc | 88 | 117 | 1 |
| 312033 | Coding | 73 | 715 | ccgggttaccacatatcctg | 90 | 118 | 1 |
| 312034 | Coding | 73 | 727 | tgcccgataccaccgggtta | 73 | 119 | 1 |
| 312035 | Coding | 73 | 743 | tcaagatgacctctggtgcc | 83 | 120 | 1 |
| 312036 | Coding | 73 | 753 | cgcatccaattcaagatgac | 83 | 121 | 1 |
| 312037 | Coding | 73 | 834 | ttgcetttgaacaggatctt | 76 | 122 | 1 |
| 312038 | Coding | 73 | 839 | ggtcattgcctttgaacagg | 84 | 123 | 1 |
| 312039 | Coding | 73 | 857 | ccttcagctggtccaggtgg | 80 | 124 | 1 |
| 312040 | Coding | 73 | 863 | tgatctccttcagctggtcc | 89 | 125 | 1 |
| 312041 | Coding | 73 | 868 | cttcatgatctccttcagct | 81 | 126 | 1 |
| 312042 | Coding | 73 | 936 | tccntgtagttcttggcctc | 81 | 127 | 1 |
| 312043 | Coding | 73 | 941 | ggccttccatgtagttcttg | 87 | 128 | 1 |
| 312044 | Coding | 73 | 946 | agggaggccttccatgtagt | 86 | 129 | 1 |
| 312045 | Coding | 73 | 951 | aactcagggaggccttccat | 86 | 130 | 1 |
| 312046 | Coding | 73 | 1050 | gtcacccgctgttccgcatc | 78 | 131 | 1 |
| 312047 | Coding | 73 | 1060 | ctcagctgctgtcaeccgct | 88 | 132 | 1 |
| 312048 | Coding | 73 | 1092 | cgangggactcaaagtatgg | 60 | 133 | 1 |
| 312049 | Coding | 73 | 1097 | tgtcecgaagggactcaaag | 39 | 134 | 1 |
| 312050 | Coding | 73 | 1107 | tcatcctcagtgtcecgaag | 87 | 135 | 1 |
| 312051 | Coding | 73 | 1164 | tccteaagggtgcggtctac | 78 | 136 | 1 |
| 312052 | Coding | 73 | 1169 | tccattcctcaagggtgcgg | 89 | 137 | 1 |
| 312053 | Coding | 73 | 1174 | acgcttccattcctcaaggg | 87 | 138 | 1 |
| 312054 | Coding | 73 | 1230 | actctggctcctagctgcct | 82 | 139 | 1 |
| 312055 | Coding | 73 | 1235 | ttggaactctggctcctagc | 87 | 140 | 1 |
| 312056 | Stop | 73 | 1261 | cagaggtcgtcacagagccg | 88 | 141 | 1 |
| | Codon | | | | | | |
| 312057 | 3′UTR | 73 | 1399 | agcatgtccaggtggaagac | 84 | 142 | 1 |
| 312058 | 3′UTR | 73 | 1449 | gccgaagaacaggtttggag | 90 | 143 | 1 |
| 312059 | 3′UTR | 73 | 1498 | tggacagaaagcttagatgt | 80 | 144 | 1 |
| 312060 | 3′UTR | 73 | 1503 | ggtcttggacagaaagctta | 83 | 145 | 1 |
| 312061 | 3′UTR | 73 | 1523 | ctagtcccatgttgggtagg | 85 | 146 | 1 |
| 312062 | 5′UTR | 72 | 4 | tgaactctctataaccgcca | 70 | 147 | 1 |
| 312063 | 5′UTR | 72 | 290 | caccacactgcaccataggc | 87 | 148 | 1 |
| 312064 | 5′UTR | 72 | 400 | cagtgcggctgtctactgca | 66 | 149 | 1 |
| 312065 | 3′UTR | 72 | 1709 | ctggeacttgtctcctaagc | 86 | 150 | 1 |
| 312066 | Coding | 74 | 25 | gcctgaactctctataaccg | 66 | 151 | 1 |
| 312067 | Coding | 74 | 642 | taggattctgtggatgacac | 64 | 152 | 1 |
| 312068 | Coding | 75 | 455 | gtctgtgaagtcgtccagag | 82 | 153 | 1 |
| 312069 | Coding | 75 | 817 | ttcaatccctccagtgtttc | 7 | 154 | 1 |
| 312070 | 5′UTR | 76 | 106 | cagtgccccacatgcaaggg | 76 | 155 | 1 |
| 312071 | 5′UTR | 76 | 203 | tagaaggatacggtcattgc | 52 | 156 | 1 |
| 312072 | 5′UTR | 76 | 377 | cactgacctctgcattctgt | 71 | 157 | 1 |
| 312073 | 5′UTR | 76 | 521 | accctgagggcttgcgttgg | 83 | 158 | 1 |
| 312074 | 5′UTR | 76 | 608 | cacagctgtgggaagagcaa | 30 | 159 | 1 |
| 312075 | 3′UTR | 76 | 1204 | cttgtctcctaagctctgct | 78 | 160 | 1 |
| 312076 | Coding | 77 | 495 | cacttaggctacaccaatgg | 52 | 161 | 1 |

As shown in Table 3, SEQ ID NOs 84, 85, 86, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 135, 136, 137, 138, 139, 140, 141, 142, 143, 144, 145, 146, 147, 148, 149, 150, 151, 152, 153, 155, 157, and 160 demonstrated at least 60% inhibition of mouse extracellular-signal-regulated kinase-6 expression in this experiment and are therefore illustrative antisense sequences. The target regions to which these antisense sequences are complementary are herein referred to as “illustrative target segments” and are therefore useful for targeting by compounds of the present invention. These target segments are shown in Table 4. The sequences represent the reverse complement of the antisense compounds shown in Table 3. “Target site” indicates the first (5′-most) nucleotide number on the particular target nucleic acid to which the oligonucleotide binds. Also shown in Table 4 is the species in which each of these target segments was found.

TABLE 4

Sequence and position of preferred target segments
identified in Extracellular-Signal-regulated kinase-6.

| | | | | | | |
|---|---|---|---|---|---|---|
| | TARGET | | | | | |
| SITE | SEQ ID | TARGET | | REV COMP | | SEQ ID |
| ID | NO | SITE | SEQUENCE | OF SEQ ID | ACTIVE IN | NO |

| | | | | | | |
|---|---|---|---|---|---|---|
| 133097 | 72 | 799 | gcaggcagacagtgagatga | 84 | M. musculus | 162 |
| 227465 | 73 | 87 | cctgccacgcagtgacccgg | 85 | M. musculus | 163 |
| 227466 | 73 | 158 | ccggctggggatgagctccc | 86 | M. musculus | 164 |
| 227468 | 73 | 189 | cgcaagggcttttaccgcca | 88 | M. musculus | 165 |
| 227469 | 73 | 195 | ggcttttaccgccaggaggt | 89 | M. musculus | 166 |
| 227470 | 73 | 200 | ttaccgccaggaggtgacca | 90 | M. musculus | 167 |
| 227471 | 73 | 209 | ggaggtgaccaaaacggcct | 91 | M. musculus | 168 |
| 227472 | 73 | 219 | aaaacggcctgggaggtgcg | 92 | M. musculus | 169 |
| 227473 | 73 | 252 | gacctgcagcccgttggctc | 93 | M. musculus | 170 |
| 227474 | 73 | 257 | gcagcccgttggctctggtg | 94 | M. musculus | 171 |
| 227475 | 73 | 267 | ggctctggtgcctatggtgc | 95 | M. musculus | 172 |
| 227476 | 73 | 272 | tggtgcctatggtgcagtgt | 96 | M. musculus | 173 |
| 227477 | 73 | 280 | atggtgcagtgtgctctgca | 97 | M. musculus | 174 |
| 227478 | 73 | 290 | gtgctctgcagtagacagcc | 98 | M. musculus | 175 |
| 227479 | 73 | 295 | ctgcagtagacagccgcact | 99 | M. musculus | 176 |
| 227480 | 73 | 300 | gtagacagccgcactggcaa | 100 | M. musculus | 177 |
| 227481 | 73 | 305 | cagccgcactggcaacaagg | 101 | M. musculus | 178 |
| 227482 | 73 | 310 | gcactggcaacaaggtggcc | 102 | M. musculus | 179 |
| 227483 | 73 | 371 | caagcgcgcctacagagagt | 103 | M. musculus | 180 |
| 227484 | 73 | 376 | gcgcctacagagagttgcgc | 104 | M. musculus | 181 |
| 227485 | 73 | 382 | acagagagttgcgcctcctc | 105 | M. musculus | 182 |
| 227486 | 73 | 389 | gttgcgcctcctcaaacaca | 106 | M. musculus | 183 |
| 227487 | 73 | 399 | ctcaaacacatgcgccacga | 107 | M. musculus | 184 |
| 227488 | 73 | 468 | gacttcacagacttctacct | 108 | M. musculus | 185 |
| 227489 | 73 | 473 | cacagacttctacctggtga | 109 | M. musculus | 186 |
| 227490 | 73 | 487 | tggtgatgccattcatgggc | 110 | M. musculus | 187 |
| 227491 | 73 | 492 | atgccattcatgggcactga | 111 | M. musculus | 188 |
| 227492 | 73 | 540 | ctgagtgaagacagaatcca | 112 | M. musculus | 189 |
| 227493 | 73 | 545 | tgaagacagaatccagtttc | 113 | M. musculus | 190 |
| 227494 | 73 | 550 | acagaatccagtttcttgtg | 114 | M. musculus | 191 |
| 227495 | 73 | 555 | atccagtttcttgtgtatca | 115 | M. musculus | 192 |
| 227496 | 73 | 681 | tttggccttgccaggcaggc | 116 | M. musculus | 193 |
| 227497 | 73 | 690 | gccaggcaggcagacagtga | 117 | M. musculus | 194 |
| 227498 | 73 | 715 | caggatatgtggtaacccgg | 118 | M. musculus | 195 |
| 227499 | 73 | 727 | taacccggtggtatcgggca | 119 | M. musculus | 196 |
| 227500 | 73 | 743 | ggcaccagaggtcatcttga | 120 | M. musculus | 197 |
| 227501 | 73 | 753 | gtcatcttgaattggatgcg | 121 | M. musculus | 198 |
| 227502 | 73 | 834 | aagatcctgttcaaaggcaa | 122 | M. musculus | 199 |
| 227503 | 73 | 839 | cctgttcaaaggcaatgacc | 123 | M. musculus | 200 |
| 227504 | 73 | 857 | ccacctggaccagctgaagg | 124 | M. musculus | 201 |
| 227505 | 73 | 863 | ggaccagctgaaggagatca | 125 | M. musculus | 202 |
| 227506 | 73 | 868 | agctgaaggagatcatgaag | 126 | M. musculus | 203 |
| 227507 | 73 | 936 | gaggccaagaactacatgga | 127 | M. musculus | 204 |
| 227508 | 73 | 941 | caagaactacatggaaggcc | 128 | M. musculus | 205 |
| 227509 | 73 | 946 | actacatggaaggcctccct | 129 | M. musculus | 206 |
| 227510 | 73 | 951 | atggaaggcctccctgagtt | 130 | M. musculus | 207 |
| 227511 | 73 | 1050 | gatgcggaacagcgggtgac | 131 | M. musculus | 208 |
| 227512 | 73 | 1060 | agcgggtgacagcagctgag | 132 | M. musculus | 209 |
| 227513 | 73 | 1092 | ccatactttgagtcccttcg | 133 | M. musculus | 210 |
| 227515 | 73 | 1107 | cttcgggacactgaggatga | 135 | M. musculus | 211 |
| 227516 | 73 | 1164 | gtagaccgcacccttgagga | 136 | M. musculus | 212 |
| 227517 | 73 | 1169 | ccgcacccttgaggaatgga | 137 | M. musculus | 213 |
| 227518 | 73 | 1174 | cccttgaggaatggaagcgt | 138 | M. musculus | 214 |
| 227519 | 73 | 1230 | aggcagctaggagccagagt | 139 | M. musculus | 215 |
| 227520 | 73 | 1235 | gctaggagccagagttccaa | 140 | M. musculus | 216 |
| 227521 | 73 | 1261 | cggctctgtgacgacctctg | 141 | M. musculus | 217 |
| 56893 | 73 | 1399 | gtcttccacctggacatgct | 142 | M. musculus | 218 |
| 227522 | 73 | 1449 | ctccaaacctgttcttcggc | 143 | M. musculus | 219 |
| 227523 | 73 | 1498 | acatctaagctttctgtcca | 144 | M. musculus | 220 |
| 227524 | 73 | 1503 | taagctttctgtccaagacc | 145 | M. musculus | 221 |
| 227525 | 73 | 1523 | cctacccaacatgggactag | 146 | M. musculus | 222 |
| 227526 | 72 | 4 | tggcggttatagagagttca | 147 | M. musculus | 223 |
| 227527 | 72 | 290 | gcctatggtgcagtgtggtg | 148 | M. musculus | 224 |
| 227528 | 72 | 400 | tgcagtagacagccgcactg | 149 | M. musculus | 225 |
| 227529 | 72 | 1709 | gcttaggagacaagtgccag | 150 | M. musculus | 226 |
| 227530 | 74 | 25 | cggttatagacagttcaggc | 151 | M. musculus | 227 |
| 227531 | 74 | 642 | gtgtcatccacagaatccta | 152 | M. musculus | 228 |
| 227532 | 75 | 455 | ctctggacgacttcacagac | 153 | M. musculus | 229 |
| 227534 | 76 | 106 | cccttgcatgtggggcactg | 155 | M. musculus | 230 |
| 227536 | 76 | 377 | acagagtgcagaggtcagtg | 157 | M. musculus | 231 |
| 227537 | 76 | 521 | ccaacgcaagccctcagggt | 158 | M. musculus | 232 |
| 227539 | 76 | 1204 | agcagagcttaggagacaag | 160 | M. musculus | 233 |

### Example 22

### Antisense inhibition of mouse extracellular-signal-regulated kinase-6 expression—dose response in b.END cells

In accordance with the present invention, three oligonucleotides targeted to mouse extracellular-signal-regulated kinase-6, ISIS 312058 (SEQ ID No: 143), ISIS 312025 (SEQ ID No: 110) and ISIS 312032 (SEQ ID No: 117), were further investigated in a dose response study. The control oligoncleotide employed in this assay is the mixed sequence 20-mer negative oligonucleotide control, ISIS 29848, NNNNNNNNNNNNNNNNNNNN, wherein N is A, T, C, or G, incorporated herein as SEQ ID NO: 71.

In the dose-response experiment, with mRNA levels as the endpoint, b.END cells were treated with ISIS 312058, ISIS 312025, and ISIS 312032 or the scrambled control oligoncleotides at doses of 6.25, 25, 75 and 150 nM oligoncleotide. Data were obtained by real-time quantitative PCR as described in other examples herein and are averaged from two experiments with mRNA levels in the treatment groups, (including the scrambled controls), being normalized to an untreated control group. The data are shown in Table 5.

TABLE 5
Inhibition of extracellular-signal-regulated kinase-6
mRNA levels by chimeric phosphorothioate oligonucleotides
having 2′-MOE wings and a deoxy gap - Dose Response

Target Reduction
% Inhibition
Dose

| ISIS NO. | 6.25 nM | 25 nM | 75 nM | 150 nM |
|---|---|---|---|---|
| 29848 | 0 | 0 | 6 | 11 |
| (control) | | | | |
| 312058 | 22 | 59 | 82 | 90 |
| 312025 | 17 | 57 | 86 | 92 |
| 312032 | 15 | 53 | 77 | 82 |

From this data, it is evident that ISIS 312058, ISIS 312025, and ISIS 312032 are capable of reducing extracellular-signal-regulated kinase-6 mRNA levels in a dose-dependent manner.

### Example 23

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the S′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2-hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group that has the following important properties. It is stable to the conditions of nucleoside phosphoramidite synthesis and oligonucleotide synthesis. However, after oligonucleotide synthesis the oligonucleotide is treated with methylamine, which not only cleaves the oligonucleotide from the solid support but also removes the acetyl groups from the orthoesters. The resulting 2-ethyl-hydroxyl substituents on the orthoester are less electron-withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 .M RNA oligonucleotide solution) and 15 μl of 5X annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

### Example 24

### Design and screening of duplexed antisense compounds targeting extracellular-signal-regulated kinase-6

In accordance with the present invention, a series of nucleic acid duplexes comprising the antisense compounds of the present invention and their complements can be designed to target extracellular-signal-regulated kinase-6. The nucleobase sequence of the antisense strand of the duplex comprises at least a portion of an oligonucleotide in Table 1. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

RNA strands of the duplex can be synthesized by methods. disclosed herein or purchased from Dharmacon Research Inc., (Lafayette, Colo.). Once synthesized, the complementary strands are annealed. The single strands are aliquoted and diluted to a concentration of 50 uM. Once diluted, 30 μL of each strand is combined with 15 μL of a 5X solution of annealing buffer. The final concentration of said buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM. This solution can be stored frozen (−20° C.) and freeze-thawed up to 5 times.

Once prepared, the duplexed antisense compounds are evaluated for their ability to modulate extracellular-signal-regulated kinase-6 expression.

When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN reagent(Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

1. A compound 8 to 80 nucleobases in length targeted to a nucleic acid molecule encoding extracellular-signal-regulated kinase-6, wherein said compound specifically hybridizes with said nucleic acid molecule encoding extracellular-signal-regulated kinase-6 and inhibits the expression of extracellular-signal-regulated kinase-6.

2. The compound according to claim 1, wherein said extracellular-signal-regulated kinase-6 is human extracellular-signal-regulated kinase-6 SEQ ID NO: 4, and wherein said compound specifically hybridizes to a sequence of at least 8 consecutive nucleotides within nucleotides 197 to 216 of SEQ ID NO: 4 and inhibits expression of said extracellular-signal-regulated kinase-6.

3. The compound according to claim 2, wherein said expression is inhibited by at least 40% as measured by a suitable assay.

4-15. (canceled)

16. The compound according to claim 1, which is an antisense oligonucleotide or chimeric oligonucleotide.

17. The compound according to claim 16, wherein the antisense oligonucleotide comprises a modification selected from the group consisting of at least one modified internucleoside linkage, at least one modified sugar moiety, and at least one modified nucleobase.

18. The compound according to claim 17, wherein the modified internucleoside linkage is a phosphorothioate linkage.

19. (canceled)

20. The compound according to claim 17, wherein the modified sugar moiety is a 2′-O-methoxyethyl sugar moiety.

21. (canceled)

22. The compound according to claim 17, wherein the modified nucleobase is a 5-methylcytosine.

23. (canceled)

24. A composition comprising the compound of claim 1 and a pharmaceutically acceptable carrier or diluent.

25. The composition according to claim 24, further comprising a colloidal dispersion system.

26. (canceled)

27. A method of inhibiting the expression of extracellular-signal-regulated kinase-6 in cells or tissues comprising contacting said cells or tissues with the compound of claim 1 so that expression of extracellular-signal-regulated kinase-6 is inhibited.

28. A method of treating an animal having a disease or condition associated with extracellular-signal-regulated kinase-6 comprising administering to said animal a therapeutically or prophylactically effective amount of the compound of claim 1 so that expression of extracellular-signal-regulated kinase-6 is inhibited.

29. The method according to claim 28, wherein the disease or condition is selected from the group consisting of a hyperproliferative disorder, an inflammatory disorder, and a neurodegenerative disorder.

30. The method according to claim 29, wherein the hyperproliferative disorder is cancer.

31-32. (canceled)

33. The method according to claim 29, wherein the neurodegenerative disorder is Alzheimer's disease.

34. A method for inhibiting angiogenesis in a mammal, the method comprising administering to a mammalian tissue a therapeutically effective amount of a compound of claim 1 in or near said tissue, whereby angiogenesis is inhibited.

35. The method according to claim 34, wherein the inhibitor prevents degradation of extracellular matrix for new blood vessel formation or prevents tubular formation of blood vessels.

36-37. (canceled)

38. The method according to claim 34, wherein said compound is selected from the group consisting of a ribozyme, an siRNA, an antisense oligonucleotide, a peptide nucleic acid, a morpholino compound and a locked nucleic acid.

39. (canceled)

40. The method according to claim 34, wherein the administration is selected from the group consisting of topical, intratracheal, intranasal, epidermal, transdermal, oral, parenteral, intravenous, intraarterial, subcutaneous, intraperitoneal or intramuscular, intracranial, intrathecal, and intraventricular.

41. The method according to claim 34, wherein at least one additional drug is administered in combination with said compound.

42. A method for preventing degradation of an extracellular matrix of mammalian tissue, the method comprising the step of inhibiting extracellular-signal-regulated kinase-6 expression in a cell of said tissue, thereby inhibiting the degradation of the extracellular matrix.

43. A method of inhibiting angiogenesis in a mammalian tissue comprising inhibiting migration of endothelial cells through the extracellular matrix by contacting said cells with a compound of claim 1.

44. A method of reducing the growth of new blood vessels supplying a tumor in mammalian tissue comprising contacting said tissue with a compound of claim 1.

45. A method for preventing tubular formation of blood vessels, the method comprising the step of inhibiting a extracellular-signal-regulated kinase-6 in a cell, thereby inhibiting the formation of blood vessels.

46. A method for treating an angiogenic disease in a mammal, the method comprising the step of administering to the mammal in need thereof a therapeutically effective amount of a compound of claim 1.

47. A method of inhibiting blood vessel formation in mammalian tissue by reducing expression of integrin β mRNA in cells of said tissue.

48. The method according to claim 47, comprising contacting said cells with an effective amount of a compound of claim 1.

49. A duplexed antisense compound comprising:
(a) a nucleobase sequence 8 to 80 nucleobases in length targeted to a nucleic acid molecule encoding extracellular-signal-regulated kinase-6 with at least one natural or modified nucleobase forming an overhang at a terminus of said sequence; and
(b) the complementary sequence of said sequence (a) having optionally at least one natural or modified nucleobase forming an overhang at a terminus of said complementary sequence;
wherein said sequences (a) and (b), when hybridized, have at least one single-stranded overhang &t at least one of terminus of said hybridized duplex, and wherein said duplex when interacted with a nucleic acid molecule encoding extracellular-signal-regulated kinase-6 can modulate the expression of said extracellular-signal-regulated kinase-6.

50-65. (canceled)

66. The compound according to claim 1, wherein said compound specifically hybridizes to a sequence of said extracellular-signal-regulated kinase-6 within at least 8 to 80 nucleobases extending 5′ of nucleobase 369 of SEQ ID NO: 4.

67. The compound according to claim 1, wherein said sequence is at least 20 nucleobases in length.

68. The compound according to claim 66, wherein said expression is inhibited by at least 40% as measured by a suitable assay.

69. The compound according to claim 66, wherein said sequence comprises nucleobases 369-388.

70. The compound according to claim 66, wherein said sequence comprises nucleobases 380-399.

71. The compound according to claim 66, wherein said sequence comprises nucleobases 394-413.

72. The compound according to claim 66, wherein said sequence comprises nucleobases 410-429.

73. The compound according to claim 66, wherein said sequence comprises nucleobases 419-438.

74. The compound according to claim 66, wherein said sequence comprises nucleobases 427-446.

75. The compound according to claim 1, wherein said compound specifically hybridizes to a sequence of said extracellular-signal-regulated kinase-6 within at least 8 to 80 nucleobases extending 5′ of nucleobase 769 of SEQ ID NO: 4.

76. The compound according to claim 75, wherein said sequence comprises nucleobases 769-788.

77. The compound according to claim 75, wherein said sequence comprises nucleobases 798-817.

78. The compound according to claim 75, wherein said sequence comprises nucleobases 807-826.

79. The compound according to claim 1, wherein said compound specifically hybridizes to a 5′ untranslated sequence of said extracellular-signal-regulated kinase-6 of SEQ ID NO: 4.

80. The compound according to claim 1, wherein said compound specifically hybridizes to a coding region sequence of said extracellular-signal-regulated kinase-6 of SEQ ID NO: 4, selected from among the sequences of Table 1.


SEQUENCE LISTING (233 sequences)

SEQ ID NO: 1 - DNA, 20 bp, Artificial Sequence
  Antisense Oligonucleotide
tccgtcatcgctcctcaggg

SEQ ID NO: 2 - DNA, 20 bp, Artificial Sequence
  Antisense Oligonucleotide
gtgcgcgcgagcccgaaatc

SEQ ID NO: 3 - DNA, 20 bp, Artificial Sequence
  Antisense Oligonucleotide
atgcattctgcccccaagga

SEQ ID NO: 4 - DNA, 1670 bp, H. sapiens
  CDS (34)...(1137)
ggctctgcggggtgggcagctcccgggcctgccatgagctctccgccgcccacccgcagtggcttttaccgccaggaggtgaccaagacggcctgggaggtgcgcgccgtgtaccgggacctgcagcccgtgggctcgggcgcctacggcgcggtgtgctcggccgtggacggccgcaccggcgctaaggttgccatcaagaagctgtatcggcccttccagtccgagctgttcgccaagctcgcctaccgcgagctgcgcctgctcaagcacatgcgccacgagaacgtgatcgggctgctggacgtattcactcctgatgagaccctggatgacttcacggacttttacctggtgatgccgttcatgggcaccgacctgggcaagctcatgaaacatgagaagctaggcgaggaccggatccagttcctcgtgtaccagatgatgaaggggctgaggtatatccacgctgccggcatcatccacagagacctgaagcccggcaacctggctgtgaacgaagactgtgagctgaagatcctggacttcggcctggccaggcaggcagacagtgagatgactgggtacgtggtgacccggtggtaccgggctcccgaggtcatcttgaattggatcgcgtacacgcagacggtggacatctggtctgtgggctgcatcatggcggagatgatcacaggcaagacgctgttcaagggcagcgaccacctggaccagctgaaggagatcatgaaggtgacggggacgcctccggctgagtttgtgcagcggctgcagagcgatgaggccaagaactacatgaagggcctccccgaattggagaagaaggattttgcctctatcctgaccaatgcaagccctctggctgtgaacctcctggagaagatgctggtgctggacgcggacatcaggttgactgcaggcgagtttctttcccatccctacttcgagtccctgcacgacacggaagatgagccccaggtccagaagtatgatgactcctttgactactttgaccgcacactggatgaatggaagcgtgttacttacaaagaggtgctcagcttcaagcctccccggcagctgggggccagggtctccaaggagacgcctctgtgaagatctctgggctccggggtggcagtgaggaccaccttcaccttccacctgagaggggactctcgttgccaccttgaccttggctggggcttgcatcccaaggcatccatcagagcagacgcccgggttccatggaccctcctccccactgccatgcctctgctctttggcgcccatcatggaggagcacctgaactttctggacaagacctctggccgacctggggatggcctctgatccctggagcagtggaacacaaaaaacaatactctcagaaacctcagagctggtggggctccagatcagccttggcctctgagccctgcctgctctgggccatgcagaggaaggacagagggtgggagcagggcaccaactcagggacatcccctctcctgggcgacgtcagtggaccttcctgcacccccagcctggaatgtaaatcagctgtgtggtgcccgcgtggctggaaggaaatagacccttttgtagctccaaaaaaaaaaaaaaaaaaaaaaaaaaaaaaaaaaaa

SEQ ID NO: 5 - DNA, 20 bp, Artificial Sequence
  PCR Primer
ctcgttgccaccttgacctt

SEQ ID NO: 6 - DNA, 16 bp, Artificial Sequence
  PCR Primer
tggaacccgggcgtct

SEQ ID NO: 7 - DNA, 24 bp, Artificial Sequence
  PCR Probe
ttgcatcccaaggcatccatcaga

SEQ ID NO: 8 - DNA, 19 bp, Artificial Sequence
  PCR Primer
gaaggtgaaggtcggagtc

SEQ ID NO: 9 - DNA, 20 bp, Artificial Sequence
  PCR Primer
gaagatggtgatgggatttc

SEQ ID NO: 10 - DNA, 20 bp, Artificial Sequence
  PCR Probe
caagcttcccgttctcagcc

SEQ ID NO: 11 - DNA, 20 bp, Artificial Sequence
  Antisense Oligonucleotide
ccttcatcatctggtacacg

SEQ ID NO: 12 - DNA, 20 bp, Artificial Sequence
  Antisense Oligonucleotide
tccttcagctggtccaggtg

SEQ ID NO: 13 - DNA, 20 bp, Artificial Sequence
  Antisense Oligonucleotide
ccaccagctctgaggtttct

SEQ ID NO: 14 - DNA, 20 bp, Artificial Sequence
  Antisense Oligonucleotide
ggagagctcatggcaggccc

SEQ ID NO: 15 - DNA, 20 bp, Artificial Sequence
  Antisense Oligonucleotide
gtggcgcatgtgcttgagca

SEQ ID NO: 16 - DNA, 20 bp, Artificial Sequence
  Antisense Oligonucleotide
cccttcatcatctggtacac

SEQ ID NO: 17 - DNA, 20 bp, Artificial Sequence
  Antisense Oligonucleotide
atccagggtctcatcaggag

SEQ ID NO: 18 - DNA, 20 bp, Artificial Sequence
  Antisense Oligonucleotide
cccggagcccagagatcttc

SEQ ID NO: 19 - DNA, 20 bp, Artificial Sequence
  Antisense Oligonucleotide
ctgtccttcctctgcatggc

SEQ ID NO: 20 - DNA, 20 bp, Artificial Sequence
  Antisense Oligonucleotide
gaactggatccggtcctcgc

SEQ ID NO: 21 - DNA, 20 bp, Artificial Sequence
  Antisense Oligonucleotide
ggccgaagtccaggatcttc

SEQ ID NO: 22 - DNA, 20 bp, Artificial Sequence
  Antisense Oligonucleotide
tgagttggtgccctgctccc

SEQ ID NO: 23 - DNA, 20 bp, Artificial Sequence
  Antisense Oligonucleotide
tctgatggatgccttgggat

SEQ ID NO: 24 - DNA, 20 bp, Artificial Sequence
  Antisense Oligonucleotide
atcatctggtacacgaggaa

SEQ ID NO: 25 - DNA, 20 bp, Artificial Sequence
  Antisense Oligonucleotide
tcgcctagcttctcatgttt

SEQ ID NO: 26 - DNA, 20 bp, Artificial Sequence
  Antisense Oligonucleotide
aggcaaaatccttcttctcc

SEQ ID NO: 27 - DNA, 20 bp, Artificial Sequence
  Antisense Oligonucleotide
tgctctgatggatgccttgg

SEQ ID NO: 28 - DNA, 20 bp, Artificial Sequence
  Antisense Oligonucleotide
gctacaaaagggtctatttc

SEQ ID NO: 29 - DNA, 20 bp, Artificial Sequence
  Antisense Oligonucleotide
ctgcctggccaggccgaagt

SEQ ID NO: 30 - DNA, 20 bp, Artificial Sequence
  Antisense Oligonucleotide
aaagaaactcgcctgcagtc

SEQ ID NO: 31 - DNA, 20 bp, Artificial Sequence
  Antisense Oligonucleotide
gaggccaaggctgatctgga

SEQ ID NO: 32 - DNA, 20 bp, Artificial Sequence
  Antisense Oligonucleotide
gtacacgaggaactggatcc

SEQ ID NO: 33 - DNA, 20 bp, Artificial Sequence
  Antisense Oligonucleotide
caccaggtaaaagtccgtga

SEQ ID NO: 34 - DNA, 20 bp, Artificial Sequence
  Antisense Oligonucleotide
tcaaagtagtcaaaggagtc

SEQ ID NO: 35 - DNA, 20 bp, Artificial Sequence
  Antisense Oligonucleotide
gggccgatacagcttcttga

SEQ ID NO: 36 - DNA, 20 bp, Artificial Sequence
  Antisense Oligonucleotide
ctggagccccaccagctctg

SEQ ID NO: 37 - DNA, 20 bp, Artificial Sequence
  Antisense Oligonucleotide
cgctgcacaaactcagccgg

SEQ ID NO: 38 - DNA, 20 bp, Artificial Sequence
  Antisense Oligonucleotide
ggcccttcatgtagttcttg

SEQ ID NO: 39 - DNA, 20 bp, Artificial Sequence
  Antisense Oligonucleotide
aggagtcatcatacttctgg

SEQ ID NO: 40 - DNA, 20 bp, Artificial Sequence
  Antisense Oligonucleotide
tctggagccccaccagctct

SEQ ID NO: 41 - DNA, 20 bp, Artificial Sequence
  Antisense Oligonucleotide
gggatgtccctgagttggtg

SEQ ID NO: 42 - DNA, 20 bp, Artificial Sequence
  Antisense Oligonucleotide
ggtgcaggaaggtccactga

SEQ ID NO: 43 - DNA, 20 bp, Artificial Sequence
  Antisense Oligonucleotide
atgtttcatgagcttgccca

SEQ ID NO: 44 - DNA, 20 bp, Artificial Sequence
  Antisense Oligonucleotide
atacctcagccccttcatca

SEQ ID NO: 45 - DNA, 20 bp, Artificial Sequence
  Antisense Oligonucleotide
gcttgcccaggtcggtgccc

SEQ ID NO: 46 - DNA, 20 bp, Artificial Sequence
  Antisense Oligonucleotide
aggtcaaggtggcaacgaga

SEQ ID NO: 47 - DNA, 20 bp, Artificial Sequence
  Antisense Oligonucleotide
tgtagttcttggcctcatcg

SEQ ID NO: 48 - DNA, 20 bp, H. sapiens
cacctggaccagctgaagga

SEQ ID NO: 49 - DNA, 20 bp, H. sapiens
agaaacctcagagctggtgg

SEQ ID NO: 50 - DNA, 20 bp, H. sapiens
tgctcaagcacatgcgccac

SEQ ID NO: 51 - DNA, 20 bp, H. sapiens
ctcctgatgagaccctggat

SEQ ID NO: 52 - DNA, 20 bp, H. sapiens
gaagatctctgggctccggg

SEQ ID NO: 53 - DNA, 20 bp, H. sapiens
gcgaggaccggatccagttc

SEQ ID NO: 54 - DNA, 20 bp, H. sapiens
gaagatcctggacttcggcc

SEQ ID NO: 55 - DNA, 20 bp, H. sapiens
gggagcagggcaccaactca

SEQ ID NO: 56 - DNA, 20 bp, H. sapiens
atcccaaggcatccatcaga

SEQ ID NO: 57 - DNA, 20 bp, H. sapiens
ttcctcgtgtaccagatgat

SEQ ID NO: 58 - DNA, 20 bp, H. sapiens
aaacatgagaagctaggcga

SEQ ID NO: 59 - DNA, 20 bp, H. sapiens
gaaatagacccttttgtagc

SEQ ID NO: 60 - DNA, 20 bp, H. sapiens
ggatccagttcctcgtgtac

SEQ ID NO: 61 - DNA, 20 bp, H. sapiens
tcaagaagctgtatcggccc

SEQ ID NO: 62 - DNA, 20 bp, H. sapiens
cagagctggtggggctccag

SEQ ID NO: 63 - DNA, 20 bp, H. sapiens
ccggctgagtttgtgcagcg

SEQ ID NO: 64 - DNA, 20 bp, H. sapiens
ccagaagtatgatgactcct

SEQ ID NO: 65 - DNA, 20 bp, H. sapiens
caccaactcagggacatccc

SEQ ID NO: 66 - DNA, 20 bp, H. sapiens
tcagtggaccttcctgcacc

SEQ ID NO: 67 - DNA, 20 bp, H. sapiens
tgggcaagctcatgaaacat

SEQ ID NO: 68 - DNA, 20 bp, H. sapiens
gggcaccgacctgggcaagc

SEQ ID NO: 69 - DNA, 20 bp, H. sapiens
tctcgttgccaccttgacct

SEQ ID NO: 70 - DNA, 20 bp, H. sapiens
cgatgaggccaagaactaca

SEQ ID NO: 71 - DNA, 20 bp, Artificial Sequence
  Antisense Oligonucleotide
nnnnnnnnnnnnnnnnnnnn

SEQ ID NO: 72 - DNA, 1929 bp, M. musculus
  CDS (512)...(1375)
ggttggcggttatagagagttcagggtggcgaccccgagagaagagcccgcaaaggaaaatctcagaggcgggcaggcgggtagccggcgcggagtacgccctgccacgcagtgacccggggcgcgcgggcggagcccctgatcccgggtccggtcctggggcgcggtgctccggctggggatgagctccccgccacccgcccgcaagggcttttaccgccaggaggtgaccaaaacggcctgggaggtgcgcgccgtgtaccaagacctgcagcccgttggctctggtgcctatggtgcagtgtggtgagcgtttgccgggcggggcggcaggcagggcaggaggcagctggcgattacggggccggttcgctagccaagccgtccgccccgcagctctgcagtagacagccgcactggcaacaaggtggccatcaagaagttgtaccggcccttccagtcggagctgtttgccaagcgcgcctacagagagttgcgcctcctcaaacacatgcgccacgagaacgtcattgggctactggatgtgttcacacctgatgagtctctggacgacttcacagacttctacctggtgatgccattcatgggcactgatctgggcaaactcatgaagcatgagaccctgagtgaagacagaatccagtttcttgtgtatcagatgttgaaggggctgaagtatatccatgcggctggtgtcatccacagagacttgaagcctggcaacctggctgtgaatgaggactgtgagctgaagatcctagactttggccttgccaggcaggcagacagtgagatgacaggatatgtggtaacccggtggtatcgggcaccagaggtcatcttgaattggatgcgctacacgcagacagtggacatttggtccgttggctgcatcatggcggagatgattactgggaagatcctgttcaaaggcaatgaccacctggaccagctgaaggagatcatgaagatcacagggacgccccctcctgagtttgttcagaagctacagagtgcagaggccaagaactacatggaaggcctccctgagttagaaaagaaggattttgcctctgtcctgaccaacgcaagccctcaggctgtgaatctcctggaaaggatgctggtgctggatgcggaacagcgggtgacagcagctgaggcgttaacccatccatactttgagtcccttcgggacactgaggatgaacccaaggcccagaaatatgacgactcctttgatgatgtagaccgcacccttgaggaatggaagcgtgtgacttacaaggaagttctcagcttcaagcctcctaggcagctaggagccagagttccaaaggagacggctctgtgacgacctctgggtggtttggggggtatccaagggaggttggctcggagcttcacggcaccttggcttcccttctctggaaaaggaatcctggttaacaccccgacagtgcctggagcttgtatcccaagtcttccacctggacatgctgtgtagacccttgaatcatgaaccctccatctccaaacctgttcttcggctttcgagtgccccagatgaccctggaagaacatctaagctttctgtccaagacccctacccaacatgggactagcctttgaattctggagttgtacatgaaatcagtattcgtgaaaaagcttcagagtgagcagagcttaggagacaagtgccagacctgagctctgctcgctctggacaatgccaaggccaactcctgagacggaatgagacagaggttttggggacactgactcagggacatcatctcttctggaagtgggtggattctcttacacccttagcctggaattcgaaccagccatttggtgttgcctaagtggctgggggcaaataaacccttttgtagatctccc

SEQ ID NO: 73 - DNA, 1553 bp, M. musculus
  CDS (168)...(1271)
gggagttcagggtggcgaccccgagagaagagcccgcaaaggaaaatctcagaggcgggcaggcgggtagccggcgcggagtacgccctgccacgcagtgacccggggcgcgcgggcggagcccctgatcccgggtccggtcctggggcgcggtgctccggctggggatgagctccccgccacccgcccgcaagggcttttaccgccaggaggtgaccaaaacggcctgggaggtgcgcgccgtgtaccaagacctgcagcccgttggctctggtgcctatggtgcagtgtgctctgcagtagacagccgcactggcaacaaggtggccatcaagaagttgtaccggcccttccagtcggagctgtttgccaagcgcgcctacagagagttgcgcctcctcaaacacatgcgccacgagaacgtcattgggctactggatgtgttcacacctgatgagtctctggacgacttcacagacttctacctggtgatgccattcatgggcactgatctgggcaaactcatgaagcatgagaccctgagtgaagacagaatccagtttcttgtgtatcagatgttgaaggggctgaagtatatccatgcggctggtgtcatccacagagacttgaagcctggcaacctggctgtgaatgaggactgtgagctgaagatcctagactttggccttgccaggcaggcagacagtgagatgacaggatatgtggtaacccggtggtatcgggcaccagaggtcatcttgaattggatgcgctacacgcagacagtggacatttggtccgttggctgcatcatggcggagatgattactgggaagatcctgttcaaaggcaatgaccacctggaccagctgaaggagatcatgaagatcacagggacgccccctcctgagtttgttcagaagctacagagtgcagaggccaagaactacatggaaggcctccctgagttagaaaagaaggattttgcctctgtcctgaccaacgcaagccctcaggctgtgaatctcctggaaaggatgctggtgctggatgcggaacagcgggtgacagcagctgaggcgttaacccatccatactttgagtcccttcgggacactgaggatgaacccaaggcccagaaatatgacgactcctttgatgatgtagaccgcacccttgaggaatggaagcgtgtgacttacaaggaagttctcagcttcaagcctcctaggcagctaggagccagagttccaaaggagacggctctgtgacgacctctgggtggtttggggggtatccaaaggaggttggctcggagcttcacggcaccttggcttcccttctctggaaaaggaatcctggttaacaccccgacagtgcctggagcttgtatcccaagtcttccacctggacatgctgtgtagacccttgaatcatgaaccctccatctccaaacctgttcttcggctttcgagtgccccagatgaccctggaagaacatctaagctttctgtccaagacccctacccaacatgggactagcctttgaattc

SEQ ID NO: 74 - DNA, 768 bp, M. musculus
agacaagggtcgggtgggagttggcggttatagagagttcaggctggcgaccccgagagaagagcccgcaaaggaaaatctcagaggcgggcaggcgggtagccggcgcggagtacgccctgccacgcagtgacccggggcgcgcgggcggagcccctgatcccgggtccggtcctggggcgcggtgctccggctggggatgagctccccgccacccgcccgcaagggcttttaccgccaggaggtgaccaaaacggcctgggaggtgcgcgccgtgtaccaagacctgcagcccgttggctctggtgcctatggtgcagtgtgctctgcagtagacagccgcactggcaacaaggtggccatcaagaagttgtaccggcccttccagtcggagctgtttgccaagcgcgcctacagagagttgcgcctcctcaaacacatgcgccacgagaacgtcattgggctactggatgtgttcacacctgatgagtctctggacgacttcacagacttctacctggtgatgccattcatgggcactgatctgggcaaactcatgaagcatgagaccctgagtgaagacagaatccagtttcttgtgtatcagatgttgaaggggctgaagtatatccatgcggctggtgtcatccacagaatcctagactttggccttgccaggcaggcagacagtgagatgacaggatatgtggtaacccggtggtatcgggcacagaggtcatcttgaattgggatgcgctacacgcagac

SEQ ID NO: 75 - DNA, 866 bp, M. musculus
  unsure 727: unknown
ttcagggtggcgaccccgagagaagagcccgcaaaggaaaatctcagaggcgggcaggcgggtagccggcgcggagtacgccctgccacgcagtgacccggggcgcgcgggcggagcccctgatcccgggtccggtcctggggcgcggtgctccggctggggatgagctccccgccacccgcccgcaagggcttttaccgccaggaggtgaccaaaacggcctgggaggtgcgcgccgtgtaccaagacctgcagcccgttggctctggtgcctatggtgcagtgtgctctgcagtagacagccgcactggcaacaaggtggccatcaagaagttgtaccggcccttccagtcggagctgtttgccaagcgcgcctacagagagttgcgcctcctcaaacacatgcgccacgagaacgtcattgggctactggatgtgttcacacctgatgagtctctggacgacttcacagactttggacatttggtccgttggctgcatcatggcggagatgattactgggaagatcctgttcaaaggcaatgaccacctggaccagctgaaggagatcatgaagatcacagggacgccccctcctgagtttgttcagaagctacagagtgcagaggccaagaactacatggaaggcctccctgagttagaaaagaaggattttgcctctgtcctgacccaacgcagcccctcaggctgtgaaatctcctggaanaggatgcctggtgctcggatgcggaaacagcggggtgaccagcagctgaaggcgttaacccctcccatacctttggagttccccttcgggaaacactggagggattgaaaccccaggggcccccgaaaatattgacgaa

SEQ ID NO: 76 - DNA, 1445 bp, M. musculus
  CDS (643)...(876)
cggacgcgtgggtatgtgcacgtctccccttcatgtatacttgtctcccacgtgtggtctctttgtgcatcccccttttcttggccttcacggatatgaatgccacccttgcatgtggggcactgatgggacttctcagtggacatttggtccgttggctgcatcatggcggagatgattactgggaagatcctgttcaaaggcaatgaccgtatccttctatgtagagcacccgttgacttcggccaagatgtgggtgctgggtgggcccccctctgtccctgcattgtattcctgaccttggtacagacctggaccagctgaaggagatcatgaagatcacagggacgccccctcctgagtttgttcagaagctacagagtgcagaggtcagtgggagtgggtggtgggctggacttgggcttgggggctggcctatcacccttttttccctcacaggccaagaactacatggaaggcctccctgagttagaaaagaaggattttgcctctgtcctgaccaacgcaagccctcagggtaccgctggatggggtgaaggtctcagggtactgggtgagtatgtcccctcctgtggcttggcctgaattgctcttcccacagctgtgaatctcctggaaaggatgctggtgctggatgcggaacagcgggtgacagcagctgaggcgttaacccatccatactttgagtcccttcgggacactgaggatgaacccaaggcccagaaatatgacgactcctttgatgatgtagaccgcacccttgaggaatggaagcgtgtgacttacaaggaagttctcagcttcaagcctcctaggcagctaggagccagagttccaaaggagacggctctgtgacgacctctgggtggtttggggggtatccaaaggaggttggctcggagcttcacggcaccttggcttcccttctctggaaaaggaatcctggttaacaccccgacagtgcctggagcttgtatcccaagtcttccacctggacatgctgtgtagacccttgaatcatgaaccctccatctccaaacctgttcttcggctttcgagtgccccagatgaccctggaagaacatctaagctttctgtccaagacccctacccaacatgggactagcctttgaattctggagttgtacatgaaatcagtattcgtgaaaaagcttcagagtgagcagagcttaggagacaagtgccagacctgagctctgctcgctctggacaatgccaaggccaactcctgagacggaatgagacagaggttttggggacactgactcagggacatcatctcttctggaagtgggtggattctcttacacccttagcctggaattcgaaccagccatttggtgttgcctaagtggctgggggcaaataaacccttttgtagatctcccaaaaaaaaaaaaaaa

SEQ ID NO: 77 - DNA, 581 bp, M. musculus
ctctgggtggtttggggggtatccaaaggaggttggctcggagcttcacggcaccttggcttcccttctctggaaaaggaatcctggttaacaccccgacagtgcctggagcttgtatcccaagtcttccacctggacatgctgtgtagacccttgaatcatgaaccctccatctccaaacctgttcttcggctttcgagtgccccagatgaccctggaagaacatctaagctttctgtccaagacccctacccaacatgggactagcctttgaattctggagttgtacatgaaatcagtattcgtgaaaaagcttcagagtgagcagagcttaggagacaagtgccagacctgagctctgctcgctctggacaatgccaaggccaactcctgagacggaatgagacagaggttaggggacactgactcagggacatcatctcttctggaagtgggtggattctcttacacccttagcctggaattcgaaccagccattggtgtagcctaagtggctgggggctaataaaacctacagtagatctccaataaatatcaaaaatataatttacaaactaatc

SEQ ID NO: 78 - DNA, 24 bp, Artificial Sequence
  PCR Primer
tctctggaaaaggaatcctggtta

SEQ ID NO: 79 - DNA, 22 bp, Artificial Sequence
  PCR Primer
ccaggtggaagacttgggatac

SEQ ID NO: 80 - DNA, 21 bp, Artificial Sequence
  PCR Probe
caccccgacagtgcctggagc

SEQ ID NO: 81 - DNA, 20 bp, Artificial Sequence
  PCR Primer
ggcaaattcaacggcacagt

SEQ ID NO: 82 - DNA, 20 bp, Artificial Sequence
  PCR Primer
gggtctcgctcctggaagat

SEQ ID NO: 83 - DNA, 27 bp, Artificial Sequence
  PCR Probe
aaggccgagaatgggaagcttgtcatc

SEQ ID NO: 84 - DNA, 20 bp, Artificial Sequence
  Antisense Oligonucleotide
tcatctcactgtctgcctgc

SEQ ID NO: 85 - DNA, 20 bp, Artificial Sequence
  Antisense Oligonucleotide
ccgggtcactgcgtggcagg

SEQ ID NO: 86 - DNA, 20 bp, Artificial Sequence
  Antisense Oligonucleotide
gggagctcatccccagccgg

SEQ ID NO: 87 - DNA, 20 bp, Artificial Sequence
  Antisense Oligonucleotide
gtaaaagcccttgcgggcgg

SEQ ID NO: 88 - DNA, 20 bp, Artificial Sequence
  Antisense Oligonucleotide
tggcggtaaaagcccttgcg

SEQ ID NO: 89 - DNA, 20 bp, Artificial Sequence
  Antisense Oligonucleotide
acctcctggcggtaaaagcc

SEQ ID NO: 90 - DNA, 20 bp, Artificial Sequence
  Antisense Oligonucleotide
tggtcacctcctggcggtaa

SEQ ID NO: 91 - DNA, 20 bp, Artificial Sequence
  Antisense Oligonucleotide
aggccgttttggtcacctcc

SEQ ID NO: 92 - DNA, 20 bp, Artificial Sequence
  Antisense Oligonucleotide
cgcacctcccaggccgtttt

SEQ ID NO: 93 - DNA, 20 bp, Artificial Sequence
  Antisense Oligonucleotide
gagccaacgggctgcaggtc

SEQ ID NO: 94 - DNA, 20 bp, Artificial Sequence
  Antisense Oligonucleotide
caccagagccaacgggctgc

SEQ ID NO: 95 - DNA, 20 bp, Artificial Sequence
  Antisense Oligonucleotide
gcaccataggcaccagagcc

SEQ ID NO: 96 - DNA, 20 bp, Artificial Sequence
  Antisense Oligonucleotide
acactgcaccataggcacca

SEQ ID NO: 97 - DNA, 20 bp, Artificial Sequence
  Antisense Oligonucleotide
tgcagagcacactgcaccat

SEQ ID NO: 98 - DNA, 20 bp, Artificial Sequence
  Antisense Oligonucleotide
ggctgtctactgcagagcac

SEQ ID NO: 99 - DNA, 20 bp, Artificial Sequence
  Antisense Oligonucleotide
agtgcggctgtctactgcag

SEQ ID NO: 100 - DNA, 20 bp, Artificial Sequence
  Antisense Oligonucleotide
ttgccagtgcggctgtctac

SEQ ID NO: 101 - DNA, 20 bp, Artificial Sequence
  Antisense Oligonucleotide
ccttgttgccagtgcggctg

SEQ ID NO: 102 - DNA, 20 bp, Artificial Sequence
  Antisense Oligonucleotide
ggccaccttgttgccagtgc

SEQ ID NO: 103 - DNA, 20 bp, Artificial Sequence
  Antisense Oligonucleotide
actctctgtaggcgcgcttg

SEQ ID NO: 104 - DNA, 20 bp, Artificial Sequence
  Antisense Oligonucleotide
gcgcaactctctgtaggcgc

SEQ ID NO: 105 - DNA, 20 bp, Artificial Sequence
  Antisense Oligonucleotide
gaggaggcgcaactctctgt

SEQ ID NO: 106 - DNA, 20 bp, Artificial Sequence
  Antisense Oligonucleotide
tgtgtttgaggaggcgcaac

SEQ ID NO: 107 - DNA, 20 bp, Artificial Sequence
  Antisense Oligonucleotide
tcgtggcgcatgtgtttgag

SEQ ID NO: 108 - DNA, 20 bp, Artificial Sequence
  Antisense Oligonucleotide
aggtagaagtctgtgaagtc

SEQ ID NO: 109 - DNA, 20 bp, Artificial Sequence
  Antisense Oligonucleotide
tcaccaggtagaagtctgtg

SEQ ID NO: 110 - DNA, 20 bp, Artificial Sequence
  Antisense Oligonucleotide
gcccatgaatggcatcacca

SEQ ID NO: 111 - DNA, 20 bp, Artificial Sequence
  Antisense Oligonucleotide
tcagtgcccatgaatggcat

SEQ ID NO: 112 - DNA, 20 bp, Artificial Sequence
  Antisense Oligonucleotide
tggattctgtcttcactcag

SEQ ID NO: 113 - DNA, 20 bp, Artificial Sequence
  Antisense Oligonucleotide
gaaactggattctgtcttca

SEQ ID NO: 114 - DNA, 20 bp, Artificial Sequence
  Antisense Oligonucleotide
cacaagaaactggattctgt

SEQ ID NO: 115 - DNA, 20 bp, Artificial Sequence
  Antisense Oligonucleotide
tgatacacaagaaactggat

SEQ ID NO: 116 - DNA, 20 bp, Artificial Sequence
  Antisense Oligonucleotide
gcctgcctggcaaggccaaa

SEQ ID NO: 117 - DNA, 20 bp, Artificial Sequence
  Antisense Oligonucleotide
tcactgtctgcctgcctggc

SEQ ID NO: 118 - DNA, 20 bp, Artificial Sequence
  Antisense Oligonucleotide
ccgggttaccacatatcctg

SEQ ID NO: 119 - DNA, 20 bp, Artificial Sequence
  Antisense Oligonucleotide
tgcccgataccaccgggtta

SEQ ID NO: 120 - DNA, 20 bp, Artificial Sequence
  Antisense Oligonucleotide
tcaagatgacctctggtgcc

SEQ ID NO: 121 - DNA, 20 bp, Artificial Sequence
  Antisense Oligonucleotide
cgcatccaattcaagatgac

SEQ ID NO: 122 - DNA, 20 bp, Artificial Sequence
  Antisense Oligonucleotide
ttgcctttgaacaggatctt

SEQ ID NO: 123 - DNA, 20 bp, Artificial Sequence
  Antisense Oligonucleotide
ggtcattgcctttgaacagg

SEQ ID NO: 124 - DNA, 20 bp, Artificial Sequence
  Antisense Oligonucleotide
ccttcagctggtccaggtgg

SEQ ID NO: 125 - DNA, 20 bp, Artificial Sequence
  Antisense Oligonucleotide
tgatctccttcagctggtcc

SEQ ID NO: 126 - DNA, 20 bp, Artificial Sequence
  Antisense Oligonucleotide
cttcatgatctccttcagct

SEQ ID NO: 127 - DNA, 20 bp, Artificial Sequence
  Antisense Oligonucleotide
tccatgtagttcttggcctc

SEQ ID NO: 128 - DNA, 20 bp, Artificial Sequence
  Antisense Oligonucleotide
ggccttccatgtagttcttg

SEQ ID NO: 129 - DNA, 20 bp, Artificial Sequence
  Antisense Oligonucleotide
agggaggccttccatgtagt

SEQ ID NO: 130 - DNA, 20 bp, Artificial Sequence
  Antisense Oligonucleotide
aactcagggaggccttccat

SEQ ID NO: 131 - DNA, 20 bp, Artificial Sequence
  Antisense Oligonucleotide
gtcacccgctgttccgcatc

SEQ ID NO: 132 - DNA, 20 bp, Artificial Sequence
  Antisense Oligonucleotide
ctcagctgctgtcacccgct

SEQ ID NO: 133 - DNA, 20 bp, Artificial Sequence
  Antisense Oligonucleotide
cgaagggactcaaagtatgg

SEQ ID NO: 134 - DNA, 20 bp, Artificial Sequence
  Antisense Oligonucleotide
tgtcccgaagggactcaaag

SEQ ID NO: 135 - DNA, 20 bp, Artificial Sequence
  Antisense Oligonucleotide
tcatcctcagtgtcccgaag

SEQ ID NO: 136 - DNA, 20 bp, Artificial Sequence
  Antisense Oligonucleotide
tcctcaagggtgcggtctac

SEQ ID NO: 137 - DNA, 20 bp, Artificial Sequence
  Antisense Oligonucleotide
tccattcctcaagggtgcgg

SEQ ID NO: 138 - DNA, 20 bp, Artificial Sequence
  Antisense Oligonucleotide
acgcttccattcctcaaggg

SEQ ID NO: 139 - DNA, 20 bp, Artificial Sequence
  Antisense Oligonucleotide
actctggctcctagctgcct

SEQ ID NO: 140 - DNA, 20 bp, Artificial Sequence
  Antisense Oligonucleotide
ttggaactctggctcctagc

SEQ ID NO: 141 - DNA, 20 bp, Artificial Sequence
  Antisense Oligonucleotide
cagaggtcgtcacagagccg

SEQ ID NO: 142 - DNA, 20 bp, Artificial Sequence
  Antisense Oligonucleotide
agcatgtccaggtggaagac

SEQ ID NO: 143 - DNA, 20 bp, Artificial Sequence
  Antisense Oligonucleotide
gccgaagaacaggtttggag

SEQ ID NO: 144 - DNA, 20 bp, Artificial Sequence
  Antisense Oligonucleotide
tggacagaaagcttagatgt

SEQ ID NO: 145 - DNA, 20 bp, Artificial Sequence
  Antisense Oligonucleotide
ggtcttggacagaaagctta

SEQ ID NO: 146 - DNA, 20 bp, Artificial Sequence
  Antisense Oligonucleotide
ctagtcccatgttgggtagg

SEQ ID NO: 147 - DNA, 20 bp, Artificial Sequence
  Antisense Oligonucleotide
tgaactctctataaccgcca

SEQ ID NO: 148 - DNA, 20 bp, Artificial Sequence
  Antisense Oligonucleotide
caccacactgcaccataggc

SEQ ID NO: 149 - DNA, 20 bp, Artificial Sequence
  Antisense Oligonucleotide
cagtgcggctgtctactgca

SEQ ID NO: 150 - DNA, 20 bp, Artificial Sequence
  Antisense Oligonucleotide
ctggcacttgtctcctaagc

SEQ ID NO: 151 - DNA, 20 bp, Artificial Sequence
  Antisense Oligonucleotide
gcctgaactctctataaccg

SEQ ID NO: 152 - DNA, 20 bp, Artificial Sequence
  Antisense Oligonucleotide
taggattctgtggatgacac

SEQ ID NO: 153 - DNA, 20 bp, Artificial Sequence
  Antisense Oligonucleotide
gtctgtgaagtcgtccagag

SEQ ID NO: 154 - DNA, 20 bp, Artificial Sequence
  Antisense Oligonucleotide
ttcaatccctccagtgtttc

SEQ ID NO: 155 - DNA, 20 bp, Artificial Sequence
  Antisense Oligonucleotide
cagtgccccacatgcaaggg

SEQ ID NO: 156 - DNA, 20 bp, Artificial Sequence
  Antisense Oligonucleotide
tagaaggatacggtcattgc

SEQ ID NO: 157 - DNA, 20 bp, Artificial Sequence
  Antisense Oligonucleotide
cactgacctctgcactctgt

SEQ ID NO: 158 - DNA, 20 bp, Artificial Sequence
  Antisense Oligonucleotide
accctgagggcttgcgttgg

SEQ ID NO: 159 - DNA, 20 bp, Artificial Sequence
  Antisense Oligonucleotide
cacagctgtgggaagagcaa

SEQ ID NO: 160 - DNA, 20 bp, Artificial Sequence
  Antisense Oligonucleotide
cttgtctcctaagctctgct

SEQ ID NO: 161 - DNA, 20 bp, Artificial Sequence
  Antisense Oligonucleotide
cacttaggctacaccaatgg

SEQ ID NO: 162 - DNA, 20 bp, M. musculus
gcaggcagacagtgagatga

SEQ ID NO: 163 - DNA, 20 bp, M. musculus
cctgccacgcagtgacccgg

SEQ ID NO: 164 - DNA, 20 bp, M. musculus
ccggctggggatgagctccc

SEQ ID NO: 165 - DNA, 20 bp, M. musculus
cgcaagggcttttaccgcca

SEQ ID NO: 166 - DNA, 20 bp, M. musculus
ggcttttaccgccaggaggt

SEQ ID NO: 167 - DNA, 20 bp, M. musculus
ttaccgccaggaggtgacca

SEQ ID NO: 168 - DNA, 20 bp, M. musculus
ggaggtgaccaaaacggcct

SEQ ID NO: 169 - DNA, 20 bp, M. musculus
aaaacggcctgggaggtgcg

SEQ ID NO: 170 - DNA, 20 bp, M. musculus
gacctgcagcccgttggctc

SEQ ID NO: 171 - DNA, 20 bp, M. musculus
gcagcccgttggctctggtg

SEQ ID NO: 172 - DNA, 20 bp, M. musculus
ggctctggtgcctatggtgc

SEQ ID NO: 173 - DNA, 20 bp, M. musculus
tggtgcctatggtgcagtgt

SEQ ID NO: 174 - DNA, 20 bp, M. musculus
atggtgcagtgtgctctgca

SEQ ID NO: 175 - DNA, 20 bp, M. musculus
gtgctctgcagtagacagcc

SEQ ID NO: 176 - DNA, 20 bp, M. musculus
ctgcagtagacagccgcact

SEQ ID NO: 177 - DNA, 20 bp, M. musculus
gtagacagccgcactggcaa

SEQ ID NO: 178 - DNA, 20 bp, M. musculus
cagccgcactggcaacaagg

SEQ ID NO: 179 - DNA, 20 bp, M. musculus
gcactggcaacaaggtggcc

SEQ ID NO: 180 - DNA, 20 bp, M. musculus
caagcgcgcctacagagagt

SEQ ID NO: 181 - DNA, 20 bp, M. musculus
gcgcctacagagagttgcgc

SEQ ID NO: 182 - DNA, 20 bp, M. musculus
acagagagttgcgcctcctc

SEQ ID NO: 183 - DNA, 20 bp, M. musculus
gttgcgcctcctcaaacaca

SEQ ID NO: 184 - DNA, 20 bp, M. musculus
ctcaaacacatgcgccacga

SEQ ID NO: 185 - DNA, 20 bp, M. musculus
gacttcacagacttctacct

SEQ ID NO: 186 - DNA, 20 bp, M. musculus
cacagacttctacctggtga

SEQ ID NO: 187 - DNA, 20 bp, M. musculus
tggtgatgccattcatgggc

SEQ ID NO: 188 - DNA, 20 bp, M. musculus
atgccattcatgggcactga

SEQ ID NO: 189 - DNA, 20 bp, M. musculus
ctgagtgaagacagaatcca

SEQ ID NO: 190 - DNA, 20 bp, M. musculus
tgaagacagaatccagtttc

SEQ ID NO: 191 - DNA, 20 bp, M. musculus
acagaatccagtttcttgtg

SEQ ID NO: 192 - DNA, 20 bp, M. musculus
atccagtttcttgtgtatca

SEQ ID NO: 193 - DNA, 20 bp, M. musculus
tttggccttgccaggcaggc

SEQ ID NO: 194 - DNA, 20 bp, M. musculus
gccaggcaggcagacagtga

SEQ ID NO: 195 - DNA, 20 bp, M. musculus
caggatatgtggtaacccgg

SEQ ID NO: 196 - DNA, 20 bp, M. musculus
taacccggtggtatcgggca

SEQ ID NO: 197 - DNA, 20 bp, M. musculus
ggcaccagaggtcatcttga

SEQ ID NO: 198 - DNA, 20 bp, M. musculus
gtcatcttgaattggatgcg

SEQ ID NO: 199 - DNA, 20 bp, M. musculus
aagatcctgttcaaaggcaa

SEQ ID NO: 200 - DNA, 20 bp, M. musculus
cctgttcaaaggcaatgacc

SEQ ID NO: 201 - DNA, 20 bp, M. musculus
ccacctggaccagctgaagg

SEQ ID NO: 202 - DNA, 20 bp, M. musculus
ggaccagctgaaggagatca

SEQ ID NO: 203 - DNA, 20 bp, M. musculus
agctgaaggagatcatgaag

SEQ ID NO: 204 - DNA, 20 bp, M. musculus
gaggccaagaactacatgga

SEQ ID NO: 205 - DNA, 20 bp, M. musculus
caagaactacatggaaggcc

SEQ ID NO: 206 - DNA, 20 bp, M. musculus
actacatggaaggcctccct

SEQ ID NO: 207 - DNA, 20 bp, M. musculus
atggaaggcctccctgagtt

SEQ ID NO: 208 - DNA, 20 bp, M. musculus
gatgcggaacagcgggtgac

SEQ ID NO: 209 - DNA, 20 bp, M. musculus
agcgggtgacagcagctgag

SEQ ID NO: 210 - DNA, 20 bp, M. musculus
ccatactttgagtcccttcg

SEQ ID NO: 211 - DNA, 20 bp, M. musculus
cttcgggacactgaggatga

SEQ ID NO: 212 - DNA, 20 bp, M. musculus
gtagaccgcacccttgagga

SEQ ID NO: 213 - DNA, 20 bp, M. musculus
ccgcacccttgaggaatgga

SEQ ID NO: 214 - DNA, 20 bp, M. musculus
cccttgaggaatggaagcgt

SEQ ID NO: 215 - DNA, 20 bp, M. musculus
aggcagctaggagccagagt

SEQ ID NO: 216 - DNA, 20 bp, M. musculus
gctaggagccagagttccaa

SEQ ID NO: 217 - DNA, 20 bp, M. musculus
cggctctgtgacgacctctg

SEQ ID NO: 218 - DNA, 20 bp, M. musculus
gtcttccacctggacatgct

SEQ ID NO: 219 - DNA, 20 bp, M. musculus
ctccaaacctgttcttcggc

SEQ ID NO: 220 - DNA, 20 bp, M. musculus
acatctaagctttctgtcca

SEQ ID NO: 221 - DNA, 20 bp, M. musculus
taagctttctgtccaagacc

SEQ ID NO: 222 - DNA, 20 bp, M. musculus
cctacccaacatgggactag

SEQ ID NO: 223 - DNA, 20 bp, M. musculus
tggcggttatagagagttca

SEQ ID NO: 224 - DNA, 20 bp, M. musculus
gcctatggtgcagtgtggtg

SEQ ID NO: 225 - DNA, 20 bp, M. musculus
tgcagtagacagccgcactg

SEQ ID NO: 226 - DNA, 20 bp, M. musculus
gcttaggagacaagtgccag

SEQ ID NO: 227 - DNA, 20 bp, M. musculus
cggttatagagagttcaggc

SEQ ID NO: 228 - DNA, 20 bp, M. musculus
gtgtcatccacagaatccta

SEQ ID NO: 229 - DNA, 20 bp, M. musculus
ctctggacgacttcacagac

SEQ ID NO: 230 - DNA, 20 bp, M. musculus
cccttgcatgtggggcactg

SEQ ID NO: 231 - DNA, 20 bp, M. musculus
acagagtgcagaggtcagtg

SEQ ID NO: 232 - DNA, 20 bp, M. musculus
ccaacgcaagccctcagggt

SEQ ID NO: 233 - DNA, 20 bp, M. musculus
agcagagcttaggagacaag